SEQUENCE LISTING (26 sequences)

SEQ ID NO: 1 - DNA, 909 bp, Dermatophagoides pteronyssinus
  CDS (1)...(906)
cggccgagctccattaagaccttcgaggaatacaagaaagccttcaacaagagctatgccaccttcgaggacgaggaggccgcgcgcaagaacttcctggaaagcgtgaaatacgtgcagagcaacggcggggctataaatcacctgtccgacctgtctttagacgagttcaagaaccggttcctgatgagcgccgaggctttcgaacaccttaagacccagtttgatctcaacgcggagaccaacgcctgcagtatcaacggcaatgcccccgctgagattgatctgcgccagatgaggaccgtgactcccatccgcatgcaaggcggctgcgggtcttgttgggccttttcaggcgtggccgcgacagagtcggcatacctcgcgtatcggaatcagagcctggacctcgctgagcaggagctcgttgactgcgcctcccaacacggatgtcatggggatacgattcccagaggtatcgaatacatccagcataatggcgtcgtgcaggaaagctattaccgatacgtagctagggagcagtcctgccgccgtcctaacgcacagcgcttcggcatttccaattattgccagatctacccccctaatgccaacaagatcagggaggccctggcgcagacgcacagcgccatcgctgtcatcatcggaatcaaggatctggacgcattccggcactatgacgggcgcacaatcatccagcgcgacaacggatatcagccaaactaccacgcggtcaacatcgtgggttactcgaacgcccagggggtggactactggatcgtgagaaacagttgggacactaactggggcgacaacggctacggctacttcgccgccaacatcgacctgatgatgatcgaggagtacccgtacgtggtgatcctgtaa

SEQ ID NO: 2 - protein, 302 aa, Dermatophagoides pteronyssinus
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 3 - protein, 302 aa, Artificial Sequence
  C4R mutant of ProDerP1
RPSSIKTFEEYKKAFNKSYATFEDEEAARKNFLESVKYVQSNGGAINHLSDLSLDEFKNRFLMSAEAFEHLKTQFDLNAETNARSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 4 - DNA, 909 bp, Artificial Sequence
  CDS (1)...(906): C4R mutant of ProDerP1
cggccgagctccattaagaccttcgaggaatacaagaaagccttcaacaagagctatgccaccttcgaggacgaggaggccgcgcgcaagaacttcctggaaagcgtgaaatacgtgcagagcaacggcggggctataaatcacctgtccgacctgtctttagacgagttcaagaaccggttcctgatgagcgccgaggctttcgaacaccttaagacccagtttgatctcaacgcggagaccaacgcccgtagtatcaacggcaatgcccccgctgagattgatctgcgccagatgaggaccgtgactcccatccgcatgcaaggcggctgcgggtcttgttgggccttttcaggcgtggccgcgacagagtcggcatacctcgcgtatcggaatcagagcctggacctcgctgagcaggagctcgttgactgcgcctcccaacacggatgtcatggggatacgattcccagaggtatcgaatacatccagcataatggcgtcgtgcaggaaagctattaccgatacgtagctagggagcagtcctgccgccgtcctaacgcacagcgcttcggcatttccaattattgccagatctacccccctaatgccaacaagatcagggaggccctggcgcagacgcacagcgccatcgctgtcatcatcggaatcaaggatctggacgcattccggcactatgacgggcgcacaatcatccagcgcgacaacggatatcagccaaactaccacgcggtcaacatcgtgggttactcgaacgcccagggggtggactactggatcgtgagaaacagttgggacactaactggggcgacaacggctacggctacttcgccgccaacatcgacctgatgatgatcgaggagtacccgtacgtggtgatcctgtaa

SEQ ID NO: 5 - protein, 302 aa, Artificial Sequence
  C31R mutant of ProDerP1
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGRGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 6 - DNA, 909 bp, Artificial Sequence
  CDS (1)...(906): C31R mutant of ProDerP1
cggccgagctccattaagaccttcgaggaatacaagaaagccttcaacaagagctatgccaccttcgaggacgaggaggccgcgcgcaagaacttcctggaaagcgtgaaatacgtgcagagcaacggcggggctataaatcacctgtccgacctgtctttagacgagttcaagaaccggttcctgatgagcgccgaggctttcgaacaccttaagacccagtttgatctcaacgcggagaccaacgcctgcagtatcaacggcaatgcccccgctgagattgatctgcgccagatgaggaccgtgactcccatccgcatgcaaggcggccgtgggtcttgttgggccttttcaggcgtggccgcgacagagtcggcatacctcgcgtatcggaatcagagcctggacctcgctgagcaggagctcgttgactgcgcctcccaacacggatgtcatggggatacgattcccagaggtatcgaatacatccagcataatggcgtcgtgcaggaaagctattaccgatacgtagctagggagcagtcctgccgccgtcctaacgcacagcgcttcggcatttccaattattgccagatctacccccctaatgccaacaagatcagggaggccctggcgcagacgcacagcgccatcgctgtcatcatcggaatcaaggatctggacgcattccggcactatgacgggcgcacaatcatccagcgcgacaacggatatcagccaaactaccacgcggtcaacatcgtgggttactcgaacgcccagggggtggactactggatcgtgagaaacagttgggacactaactggggcgacaacggctacggctacttcgccgccaacatcgacctgatgatgatcgaggagtacccgtacgtggtgatcctgtaa

SEQ ID NO: SEQ ID N   O 7 - protein, 302 aa, Artificial Sequence
  C65R mutant of ProDerP1
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDRASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 8 - DNA, 909 bp, Artificial Sequence
  CDS (1)...(906): C65R mutant of ProDerP1
cggccgagctccattaagaccttcgaggaatacaagaaagccttcaacaagagctatgccaccttcgaggacgaggaggccgcgcgcaagaacttcctggaaagcgtgaaatacgtgcagagcaacggcggggctataaatcacctgtccgacctgtctttagacgagttcaagaaccggttcctgatgagcgccgaggctttcgaacaccttaagacccagtttgatctcaacgcggagaccaacgcctgcagtatcaacggcaatgcccccgctgagattgatctgcgccagatgaggaccgtgactcccatccgcatgcaaggcggctgcgggtcttgttgggccttttcaggcgtggccgcgacagagtcggcatacctcgcgtatcggaatcagagcctggacctcgctgagcaggagctcgttgaccgtgcctcccaacacggatgtcatggggatacgattcccagaggtatcgaatacatccagcataatggcgtcgtgcaggaaagctattaccgatacgtagctagggagcagtcctgccgccgtcctaacgcacagcgcttcggcatttccaattattgccagatctacccccctaatgccaacaagatcagggaggccctggcgcagacgcacagcgccatcgctgtcatcatcggaatcaaggatctggacgcattccggcactatgacgggcgcacaatcatccagcgcgacaacggatatcagccaaactaccacgcggtcaacatcgtgggttactcgaacgcccagggggtggactactggatcgtgagaaacagttgggacactaactggggcgacaacggctacggctacttcgccgccaacatcgacctgatgatgatcgaggagtacccgtacgtggtgatcctgtaa

SEQ ID NO: 9 - protein, 302 aa, Artificial Sequence
  C71R mutant of ProDerP1
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGRHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 10 - DNA, 909 bp, Artificial Sequence
  CDS (1)...(906): C71R mutant of ProDerP1
cggccgagctccattaagaccttcgaggaatacaagaaagccttcaacaagagctatgccaccttcgaggacgaggaggccgcgcgcaagaacttcctggaaagcgtgaaatacgtgcagagcaacggcggggctataaatcacctgtccgacctgtctttagacgagttcaagaaccggttcctgatgagcgccgaggctttcgaacaccttaagacccagtttgatctcaacgcggagaccaacgcctgcagtatcaacggcaatgcccccgctgagattgatctgcgccagatgaggaccgtgactcccatccgcatgcaaggcggctgcgggtcttgttgggccttttcaggcgtggccgcgacagagtcggcatacctcgcgtatcggaatcagagcctggacctcgctgagcaggagctcgttgactgcgcctcccaacacggacgtcatggggatacgattcccagaggtatcgaatacatccagcataatggcgtcgtgcaggaaagctattaccgatacgtagctagggagcagtcctgccgccgtcctaacgcacagcgcttcggcatttccaattattgccagatctacccccctaatgccaacaagatcagggaggccctggcgcagacgcacagcgccatcgctgtcatcatcggaatcaaggatctggacgcattccggcactatgacgggcgcacaatcatccagcgcgacaacggatatcagccaaactaccacgcggtcaacatcgtgggttactcgaacgcccagggggtggactactggatcgtgagaaacagttgggacactaactggggcgacaacggctacggctacttcgccgccaacatcgacctgatgatgatcgaggagtacccgtacgtggtgatcctgtaa

SEQ ID NO: 11 - protein, 302 aa, Artificial Sequence
  C103R mutant of ProDerP1
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSRRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 12 - DNA, 909 bp, Artificial Sequence
  CDS (1)...(906): C103R mutant of ProDerP1
cggccgagctccattaagaccttcgaggaatacaagaaagccttcaacaagagctatgccaccttcgaggacgaggaggccgcgcgcaagaacttcctggaaagcgtgaaatacgtgcagagcaacggcggggctataaatcacctgtccgacctgtctttagacgagttcaagaaccggttcctgatgagcgccgaggctttcgaacaccttaagacccagtttgatctcaacgcggagaccaacgcctgcagtatcaacggcaatgcccccgctgagattgatctgcgccagatgaggaccgtgactcccatccgcatgcaaggcggctgcgggtcttgttgggccttttcaggcgtggccgcgacagagtcggcatacctcgcgtatcggaatcagagcctggacctcgctgagcaggagctcgttgactgcgcctcccaacacggatgtcatggggatacgattcccagaggtatcgaatacatccagcataatggcgtcgtgcaggaaagctattaccgatacgtagctagggagcagtcccgtcgccgtcctaacgcacagcgcttcggcatttccaattattgccagatctacccccctaatgccaacaagatcagggaggccctggcgcagacgcacagcgccatcgctgtcatcatcggaatcaaggatctggacgcattccggcactatgacgggcgcacaatcatccagcgcgacaacggatatcagccaaactaccacgcggtcaacatcgtgggttactcgaacgcccagggggtggactactggatcgtgagaaacagttgggacactaactggggcgacaacggctacggctacttcgccgccaacatcgacctgatgatgatcgaggagtacccgtacgtggtgatcctgtaa

SEQ ID NO: 13 - protein, 302 aa, Artificial Sequence
  C117R mutant of ProDerP1
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYRQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 14 - DNA, 909 bp, Artificial Sequence
  CDS (1)...(906): C117R mutant of ProDerP1
cggccgagctccattaagaccttcgaggaatacaagaaagccttcaacaagagctatgccaccttcgaggacgaggaggccgcgcgcaagaacttcctggaaagcgtgaaatacgtgcagagcaacggcggggctataaatcacctgtccgacctgtctttagacgagttcaagaaccggttcctgatgagcgccgaggctttcgaacaccttaagacccagtttgatctcaacgcggagaccaacgcctgcagtatcaacggcaatgcccccgctgagattgatctgcgccagatgaggaccgtgactcccatccgcatgcaaggcggctgcgggtcttgttgggccttttcaggcgtggccgcgacagagtcggcatacctcgcgtatcggaatcagagcctggacctcgctgagcaggagctcgttgactgcgcctcccaacacggatgtcatggggatacgattcccagaggtatcgaatacatccagcataatggcgtcgtgcaggaaagctattaccgatacgtagctagggagcagtcctgccgccgtcctaacgcacagcgcttcggcatttccaattatcgtcagatctacccccctaatgccaacaagatcagggaggccctggcgcagacgcacagcgccatcgctgtcatcatcggaatcaaggatctggacgcattccggcactatgacgggcgcacaatcatccagcgcgacaacggatatcagccaaactaccacgcggtcaacatcgtgggttactcgaacgcccagggggtggactactggatcgtgagaaacagttgggacactaactggggcgacaacggctacggctacttcgccgccaacatcgacctgatgatgatcgaggagtacccgtacgtggtgatcctgtaa

SEQ ID NO: 15 - DNA, 108 bp, Artificial Sequence
  Primer
ttaagacccagtttgatctcaacgcggagaccaacgcccgtatcaacggcaatgcccccgctgagattgatctgcgccagatgaggaccgtgactcccatccgcatgc

SEQ ID NO: 16 - DNA, 103 bp, Artificial Sequence
  Primer
cggatgggagtcacggtcctcatctggcgcagatcaatctcagcgggggcattgccgttgatactacgggcgttggtctccgcgttgagatcgaaactgggtc

SEQ ID NO: 17 - DNA, 92 bp, Artificial Sequence
  Primer
caaggcggccgtgggtcttgttgggccttttcaggcgtggccgcgacagagtcggcatacctcgcgtatcggaatcagagcctggacctcgc

SEQ ID NO: 18 - DNA, 99 bp, Artificial Sequence
  Primer
tcagcgaggtccaggctctgattccgatacgcgaggtatgccgactctgtcgcggccacgcctgaaaaggcccaacaagacccacggccgccttgcatg

SEQ ID NO: 19 - DNA, 83 bp, Artificial Sequence
  Primer
tgagcaggagctcgttgaccgtgcctcccaacacggatgtcatggggatacgattcccagaggtatcgaatacatccagcata

SEQ ID NO: 20 - DNA, 77 bp, Artificial Sequence
  Primer
ctggatgtattcgatacctctgggaatcgtatcccccatgacatccgtgttgggaggcacggtcaacgcgctcctgc

SEQ ID NO: 21 - DNA, 29 bp, Artificial Sequence
  Primer
actgacaggcctcggccgagctccattaa

SEQ ID NO: 22 - DNA, 29 bp, Artificial Sequence
  Primer
cagtcacctaggtctagactcgaggggat

SEQ ID NO: 23 - DNA, 25 bp, Artificial Sequence
  Primer
ggctttcgaacaccttaagacccag

SEQ ID NO: 24 - DNA, 27 bp, Artificial Sequence
  Primer
gctccctagctacgtatcggtaatagc

SEQ ID NO: 25 - DNA, 29 bp, Artificial Sequence
  Primer
cctcgcgtatcggcaacagagcctggacc

SEQ ID NO: 26 - DNA, 29 bp, Artificial Sequence
  Primer
ggtccaggctctgttgccgatacgcgagg

(19) United States
(12) Patent Application Publication
(10) Pub. No.: US 2004/0234538 A1
(43) Pub. Date: Nov. 25, 2004

(54) Derp1 and proderp1 allergen derivatives

(76) Inventors: Alex Bollen, Gosselies (BE); Alain Jacquet, Gosselies (BE); Mauro Magi, Gosselies (BE)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/486,910

(22) PCT Filed: Aug. 15, 2002
(86) PCT No.: PCT/EP02/09122

(30) Foreign Application Priority Data
Aug. 17, 2001 (GB) 0120150.8

(51) Int. Cl. A61K 39/00; C07H 21/04; C07K 14/435

(52) U.S. Cl. 424/185.1; 530/350; 435/69.3; 435/348; 435/320.1; 536/23.5

(57) ABSTRACT

The present invention provides a novel treatment for allergy comprising the provision of a recombinant DerP1/ProDerP1 allergen derivative with hypoallergenic activity. Pharmaceutical compositions comprising said mutant allergens which stimulate a Th1-type immune response in allergic or naïve individuals thereby reducing the potential for an allergic response upon contact with the wild-type allergen, are also provided.

The present invention relates to novel prophylactic and therapeutic formulations, said formulations being effective in the prevention and/or the reduction of allergic responses to specific allergens. Further this invention relates to hypoallergenic recombinant derivatives of the major protein allergen from Dermatophagoides pteronyssinus, allergen DerP1 and its precursor form ProDerP1. In particular the derivatives of the invention include physically modified DerP1 or ProDerP1 such as the thermally treated protein; or genetically modified recombinant DerP1 or ProDerP1 wherein one or more cystein residues' involved in disulphide bond formation have been mutated. Methods are also described for expressing and purifying the DerP1 and ProDerP1 derivatives and for formulating immunogenic compositions and vaccines.

Allergic responses in humans are common, and may be triggered by a variety of allergens. Allergic individuals are sensitised to allergens, and are characterised by the presence of high levels of allergen specific IgE in the serum, and possess allergen specific T-cell populations which produce Th2-type cytokines (IL-4, IL-5, and IL-13). Binding of IgE, in the presence of allergen, to FcεRI receptors present on the surface of mastocytes and basophils, leads to the rapid degranulation of the cells and the subsequent release of histamine, and other preformed and neoformed mediators of the inflammatory reaction. In addition to this, the stimulation of the T-cell recall response results in the production of IL-4 and IL-13, together cooperating to switch B-cell responses further towards allergen specific IgE production. For details of the generation of early and late phase allergic responses see Joost Van Neeven et al., 1996, Immunology Today, 17, 526. In non-allergic individuals, the immune response to the same antigens may additionally include Th1-type cytokines such as IFN-γ. These cytokines may prevent the onset of allergic responses by the inhibition of high levels of Th2-type immune responses, including high levels of allergen specific IgE. Importantly in this respect, is the fact that IgE synthesis may be controlled by an inhibitory feedback mechanism mediated by the binding of IgE/allergen complexes to the CD23 (FcεRII) receptor on B-cells (Luo et al., J.Immunol., 1991, 146(7), 2122-9; Yu et al., 1994, Nature, 369(6483):753-6). In systems that lack cellular bound CD23, this inhibition of IgE synthesis does not occur.

Type I allergic diseases mediated by IgE against allergens such as bronchial asthma, atopic dermatitis and perrenial rhinitis affect more than 20% of the world's population. Current strategies in the treatment of such allergic responses include means to prevent the symptomatic effects of histamine release by anti-histamine treatments and/or local administration of anti-inflammatory corticosteroids. Other strategies which are under development include those which use the hosts immune system to prevent the degranulation of the mast cells, Stanworth et al., EP 0 477 231 B1. Other forms of immunotherapy have been described (Hoyne et al., J.Exp.Med., 1993, 178, 1783-1788; Holt et al., Lancet, 1994, 344, 456-458).

While immediate as well as late symptoms can be ameliorated by pharmalogical treatment, allergen-specific immunotherapy is the only curative approach to type I allergy. However, some problems related to this method remain to be solved. First, immunotherapy is currently performed with total allergen extracts which can be heterogeneous from batch to batch. Moreover, these allergen mixtures are not designed for an individual patient's profile and may contain unwanted toxic proteins. Second, the administration of native allergens at high doses can cause severe anaphylactic reactions and therefore the optimally efficient high dose of allergen for successful immunotherapy can often not be reached. The first problem has been addressed through alternative vaccination with better characterised and more reproducible recombinant allergens as compared to allergen extracts. The second problem, namely the risk of anaphylactic reactions induced by repeated injections of allergen extracts, can be minimised through the use of recombinant “hypoallergens”, whose the IgE reactivity was altered by deletions or mutagenesis (Akdis, C A and Blaser, K, Regulation of specific immune responses by chemical and structural modifications of allergens, Int. Arch. Allergy Immunol., 2000, 121,261-269).

Formulations have been described for the treatment and prophylaxis of allergy, which provide means to down-regulate the production of IgE, as well as modifying the cell mediated response to the allergen, through a shift from a Th2 type to a Th1 type of response (as measured by the reduction of ratio of IL-4: IFN-γ producing DerP1 specific T-cells, or alternatively a reduction of the IL-5:IFN-γ ratio). This may for example be achieved through the use of recombinant allergens such as recDerP1 with reduced enzymatic activity as described in WO 99/25823. However the immunogenicity of these recombinant allergens is thought to be similar to that of wild-type ProDerP1 in terms of IgE synthesis induction.

Non-anaphylactic forms of allergens with reduced IgE-binding activity have been reported. Allergen engineering has allowed a reduction of IgE-binding capacities of the allergen proteins by site-directed mutagenesis of amino acid residues or deletions of certain amino acid sequences. In the same time, T-cell activating capacity is still conserved as T cell epitopes are maintained. This has been shown using several approaches for different allergens although with variable results. Examples have been published for the timothy grass pollen allergen Ph1 p 5b (Schramm G et al., 1999, J Immunol., 162, 2406-14), for the major house dust mite allergens Derf2 (Takai et al. 2000, Eur. J. Biochem., 267, 6650-6656), DerP2 (Smith & Chapman 1996, Mol. Immunol. 33, 399-405) and Derf1 (Takahashi K et al. 2001, Int Arch Allergy Immunol.124, 454-60). One study has reported the generation of Derf1 hypoallergens by introductions of point mutations at the level of cysteine residues involved in disulfides bridges (Takahashi K Int Arch Allergy Immunol. 2001;124(4):454-60., Takai T, Yasuhara T, Yokota T, Okumura Y). However, if wild-type ProDerf1 was successfully secreted by P. pastoris, cysteine mutants concerning intramolecular disulfide bonds were, by contrast, not secreted.

The allergens from the house dust mite Dermatophagoides pteronyssinus are one of the major causative factors associated with allergic hypersensitivity reactions. Amongst these molecules, DerP1 is a an immunodominant allergen which elicits the strongest IgE-mediated immune response (Topham et al., 1994, Protein Engineering, 7, 7, 869-894; Simpson et al., 1989, Protein Sequences and Data Analyses, 2, 17-21) and with more than 75% of allergic patients to dust mites who develop IgE directed to this allergen. Hypoallergen derived from house dust mite DerP1, and effective prophylactic as well as therapeutic vaccine against this allergen have never been described.

The present invention relates to the provision and use of recombinant derivatives of Dennatophagoides pteronyssinus DerP1 allergen or of its precursor form ProDerP1 thereafter referred to as “DerP1/ProDerP1”, with reduced allergenic activity compared to the wild-type allergen. The recombinant forms of DerP1 derivatives according to the invention, either adjuvanted recombinant proteins or plasmid encoding DerP1/ProDerP1 suitable for NAVAC, are used as prophylactic or therapeutic vaccines to induce strong preventive Th1 or to shift Th2 to Th1 immune responses. The hypoallergenic derivatives can be successfully produced in recombinant expression systems and this is also an aspect of the present invention.

DerP1 is a 30 KDa protein and has been cloned and sequenced (Chua et al., 1988, J.Exp.Med., 167, 175-182). It is known to contain 222 amino acid residues in the mature protein. The sequence of DerP1 shares 31% homology to papain, and shares more particularly homology in the enzymatically active regions, most notably the Cys34-His170 ion pair (Topham et al., supra). DerP1 is produced in the mid-gut of the mite, where its role is probably related to the digestion of food. Up to 0.2 ng or proteolytically active DerP1 is incorporated into each fecal pellet, each around 10-40 μm in diameter and, therefore, easily inspired into the human respiratory tract. Overnight storage of purified DerP1 preparations at room temperature results in almost complete loss of enzymatic activity due to autoproteolytic degradation (Machado et al., 1996, Eur.J.Immunol. 26, 2972-2980). The DerP1 encoding cDNA sequence reveals that, like many mammalian and plant proteinases, DerP1 is synthetised as an inactive preproenzyme of 320 amino acid residues which is subsequently processed into a 222-amino acid mature form (Chua et al., 1988, J.Exp.Med., 167, 175-182; Chua et al., 1993, Int. Arch Allergy Immunol 101, 364-368). The maturation of ProDerP1 is not known to date but it is thought that the allergen is processed by the cleavage of the 80-residues proregion.

The present invention provides a recombinant Dermatophagoides pteronyssinus DerP1/ProDerP1 protein allergen derivative wherein said allergen derivative has a significantly reduced allergenic activity compared to that the wild-type allergen. The allergenic activity can be impaired by several means which all aim at destructuring the protein forms by disrupting its intramolecular disulphide bridges thereby destabilising its 3-dimensional structure. Said allergen derivatives having the following advantages over the unaltered wild-type allergen: 1) increases the Th1-type aspect of the immune responses (higher IgG2a for example) in comparison to those stimulated by the wild type allergen, thereby leading to the suppression of allergic potential of the vaccinated host, 2) having reduced allergenicity while still retaining T cell reactivity, thus being more suitable for systemic administration of high doses of the immunogen, 3) will induce DerP1 specific IgG which compete with IgE for the binding of native DerP1, 4) efficiently protects against airway eosinophilia even after exposure to aerosolised allergen extract. Such derivatives are suitable for use in therapeutic and prophylactic vaccine formulations which are suitable for use in medecine and more particularly for the treatment or prevention of allergic reactions.

According to a first aspect, the present invention provides a recombinant DerP1/ProDerP1 (i.e. DerP1 or ProDerP1) allergen derivative wherein the allergenic activity has been significantly reduced, e.g. almost or completely abolished, by a physical means such as by thermally treating the protein, preferably in the presence of a reducing agent. Typically, the DerP1/ProDerP1 protein is treated during a few minutes at about 100° C. in the presence of a reducing agent. Preferably the reducing agent is beta-mercaptoethanol or DTT. Still more preferably the protein is treated during 5 minutes at about 100° C. in the presence of 50 mM beta-mercaptoethanol. This treatment has a detrimental effect on the stability of the protein conformational IgE-binding epitopes.

In a second aspect the present invention provides a recombinant DerP1/ProDerP1 protein derivative wherein the allergenic activity has been genetically impaired such as by introducing specific mutations into the encoding cDNA or the genomic DNA. Accordingly an aspect of the invention provides the genetically mutated recombinant DerP1/ProDerP1 per se. The reduction of the allergenicity of DerP1/ProDerP1 may be performed by introducing mutations into the native sequence before recombinantly producing the hypoallergenic mutants. This may be achieved by: introducing substitutions, deletions, or additions in or by altering the three dimensional structure of the protein such that the tridimensional conformation of the protein is lost. This may be achieved, amongst others, by expressing the protein in fragments, or by deleting cysteine residues involved in disulphide bridge formation, or by deleting or adding residues such that the tertiary structure of the protein is substantially altered. Preferably, mutations may be generated with the effect of altering the interaction between two cysteine residues, typically one mutation at positions 4, 31, 65, 71, 103 and 117 of the native—mature—DerP1 (which corresponds to positions 84, 111, 145, 151, 183 and 197 of ProDerP1, respectively). A mutated protein according to the invention may comprise two or more (3, 4, 5 or all 6) cysteine mutations, thereby affecting different disulphide bridges, such as mutations at positions 4 & 31, 4 & 65, 4 & 71, 4 & 103, 31 & 65, or 4 & 31 & 65, or at positions 71 & 103, 71 & 117, 103 & 117, 31 & 117, 65 & 117, or 71 & 103 & 117. Preferably the derivatives comprise one single mutation at any of the above positions. The most preferred mutation involves Cys4 (or alternatively, or in addition, Cys117 which is thought to be the disulphide bond partner of Cys4). The Cys mutations can be deletions, but are preferably substitutions for any of the other natural 19 amino acids. Preferred substitutions introduce positively charged amino acid residues to further destabilise the 3D-structure of the resulting protein. For example, preferred substitutions involve cysteine→arginine (or lysine) substitution.

Accordingly, the invention is illustrated herein by, but is not limited to, six specific mutations which are given as examples of hypoallergenic DerP1/ProDerP1 derivatives. First the allergenic activity of ProDerP1 is substantially reduced, preferably completely abrogated by substituting a cysteine residue for an arginine residue at position Cys4 of DerP1 protein sequence, and is set out in SEQ ID NO:3. Second, the allergenic activity of ProDerP1 is substantially abrogated by substituting a cysteine residue for an arginine residue at any of the following positions (calculated by reference to the sequence in mature DerP1): Cys31 of DerP1 protein sequence (SEQ ID NO:5), Cys65 (SEQ ID NO:7), Cys71 (SEQ ID NO:9), Cys103 (SEQ ID NO:11), Cys117 (SEQ ID NO:13).

Mutated versions of DerP1/ProDerP1 may be prepared by site-directed mutagenesis of the cDNA which codes for the DerP1/ProDerP1 protein by conventional methods such as those described by G. Winter et al in Nature 1982, 299, 756-758 or by Zoller and Smith 1982; Nucl. Acids Res., 10, 6487-6500, or deletion mutagenesis such as described by Chan and Smith in Nucl. Acids Res., 1984, 12, 2407-2419 or by G. Winter et al in Biochem. Soc. Trans., 1984, 12, 224-225.

The invention is not limited to the specifically disclosed sequence, but includes any hypoallergenic allergen which has been mutated to decrease or abolish its IgE-binding reactivity and/or histamine release activity, whilst retaining its T cell reactivity and/or the ability to stimulate an immune response against the wild-type allergen. The allergenic activity, and consequently the reduction in the allergenic activity, of the mutant allergens may be compared to the wild type by any of the following methods: histamine release activity or by IgE-binding reactivity, according to the method detailed in the Example section.

“Substantially reduced allergenic activity” means that the allergenic activity as measured by residual IgE-binding activity is reduced to a maximum of 50% of the activity of the native—unmodified or unmutated—protein, preferably to a maximum of 20%, more preferably to a maximum of 10%, still more preferably to a maximum of 5%, still more preferably to less than 5%. Alternatively, “substantially” also means that the histamine release activity of the mutant is reduced by at least a 100-fold factor as compared to the native protein, preferably by a factor of 1000-fold, still more preferably by a factor of 10000-fold.

The immunogenicity of the mutant allergen may be compared to that of the wild-type allergen by various immunologicals assays. The cross-reactivity of the mutant and wild-type allergens may be assayed by in vitro T-cell assays after vaccination with either mutant or wild-type allergens. Briefly, splenic T-cells isolated from vaccinated animals may be restimulated in vitro with either mutant or wild-type allergen followed by measurement of cytokine production with commercially available ELISA assays, or proliferation of allergen specific T cells may be assayed over time by incorporation of tritiated thymidine. Also the immunogenicity may be determined by ELISA assay, the details of which may be easily determined by the man skilled in the art. Briefly, two types of ELISA assay are envisaged. First, to assess the recognition of the mutant DerP1 by sera of mice immunized with the wild type DerP1; and secondly by recognition of wild type DerP1 allergen by the sera of animals immunised with the mutant allergen. Briefly, each wells will be coated with 100 ng of purified wild type or mutated DerP1 overnight at 4° C. After incubating with a blocking solution (TBS-Tween 0.1% with 1% BSA) successive dilutions of sera will be incubated at 37° C. for 1 hour. The wells are washed 5 times, and total IgG revealed by incubating with an anti-IgG antibody conjugated with Alkaline phosphatase.

A further aspect of the present invention provides an isolated nucleic acid encoding a mutated version of the DerP1/ProDerP1 allergen as disclosed herein. Preferably the nucleotide sequence is a DNA sequence and can be synthesized by standard DNA synthesis techniques, such as by enzymatic ligation as described by D. M. Roberts et al in Biochemistry 1985, 24, 5090-5098, by chemical synthesis, by in vitro enzymatic polymerization, or by a combination of these techniques. Preferably the nucleic acid sequence has a codon usage pattern that has been optimised so as to mimic the one used in the intended expression host, more preferably resembling that of highly expressed mammalian e.g. human genes. Preferred DNA sequences are codon-optimised sequences and are set out in SEQ ID NO:4, SEQ ID NO:6, SEQ ID NO:8, SEQ ID NO:10, SEQ ID NO:12 and SEQ ID NO:14.

Enzymatic polymerisation of DNA may be carried out in vitro using a DNA polymerase such as DNA polymerase I (Klenow fragment) in an appropriate buffer containing the nucleoside triphosphates dATP, dCTP, dGTP and dTTP as required at a temperature of 10°-37° C., generally in a volume of 50 ml or less. Enzymatic ligation of DNA fragments may be carried out using a DNA ligase such as T4 DNA ligase in an appropriate buffer, such as 0.05M Tris (pH 7.4), 0.01M MgCl₂, 0.01M dithiothreitol, 1 mM spermidine, 1 mM ATP and 0.1 mg/ml bovine serum albumin, at a temperature of 4° C. to ambient, generally in a volume of 50 ml or less. The chemical synthesis of the DNA polymer or fragments may be carried out by conventional phosphotriester, phosphite or phosphoramidite chemistry, using solid phase techniques such as those described in ‘Chemical and Enzymatic Synthesis of Gene Fragments—A Laboratory Manual’ (ed. H. G. Gassen and A. Lang), Verlag Chemie, Weinheim (1982), or in other scientific publications, for example M. J. Gait, H. W. D. Matthes, M. Singh, B. S. Sproat, and R. C. Titmas, Nucleic Acids Research, 1982, 10, 6243; B. S. Sproat and W. Bannwarth, Tetrahedron Letters, 1983, 24, 5771; M. D. Matteucci and M. H Caruthers, Tetrahedron Letters, 1980, 21, 719; M. D. Matteucci and M. H. Caruthers, Journal of the American Chemical Society, 1981, 103, 3185; S. P. Adams et al., Journal of the American Chemical Society, 1983, 105, 661; N. D. Sinha, J. Biernat, J. McMannus, and H. Koester, Nucleic Acids Research, 1984, 12, 4539; and H. W. D. Matthes et al., EMBO Journal, 1984, 3, 801.

Alternatively, the coding sequence can be derived from DerP1/ProDerP1 mRNA, using known techniques (e.g. reverse transcription of mRNA to generate a complementary cDNA strand), and commercially available cDNA kits.

Desirably the codon usage pattern of the nucleotide sequence is typical of highly expressed human genes. Accordingly there is provided in a particular aspect of the invention a nucleotide sequence comprising a plurality of codons together encoding the mutated DerP1/ProDerP1 protein, wherein the selection of the possible codons used for encoding the recombinant mite protein amino acid sequence has been changed to closely mimic the optimised mammalian codon usage, such that the frequency of codon usage in the resulting gene sequence is substantially the same as a mammalian gene which would encode the same protein. Codon usage patterns for mammals, including humans, can be found in the literature (see e.g. Nakamura et al. 1996, Nucleic Acids Res. 24, 214-215).

The DNA code has 4 letters (A, T, C and G) and uses these to spell three letter “codons” which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons.

Where more than one codon is available to code for a given amino acid, it has been observed that the codon usage patterns of organisms are highly non-random. Different species show a different bias in their codon selection and, furthermore, utilization of codons may be markedly different in a single species between genes which are expressed at high and low levels. This bias is different in viruses, plants, bacteria, insect and mammalian cells, and some species show a stronger bias away from a random codon selection than others. For example, humans and other mammals are less strongly biased than certain bacteria or viruses. For these reasons, there is a significant probability that a mammalian gene expressed in E. coli or a viral gene expressed in mammalian cells will have an inappropriate distribution of codons for efficient expression. However, a gene with a codon usage pattern suitable for E. coli expression may also be efficiently expressed in humans. It is believed that the presence in a heterologous DNA sequence of clusters of codons which are rarely observed in the host in which expression is to occur, is predictive of low heterologous expression levels in that host.

There are several examples where changing codons from those which are rare in the host to those which are host-preferred (“codon optimisation”) has enhanced heterologous expression levels, for example the BPV (bovine papilloma virus) late genes L1 and L2 have been codon optimised for mammalian codon usage patterns and this has been shown to give increased expression levels over the wild-type HPV sequences in mammalian (Cos-1) cell culture (Zhou et. al. J. Virol 1999. 73, 4972-4982). In this work, every BPV codon which occurred more than twice as frequently in BPV than in mammals (ratio of usage >2), and most codons with a usage ratio of >1.5 were conservatively replaced by the preferentially used mammalian codon. In WO97/31115, WO97/48370 and WO98/34640 Merck & Co., Inc.) codon optimisation of HIV genes or segments thereof has been shown to result in increased protein expression and improved immunogenicity when the codon optimised sequences are used as DNA vaccines in the host mammal for which the optimisation was tailored.

In this work, the sequences preferably consist entirely of optimised codons (except where this would introduce an undesired restriction site, intron splice site etc.) because each D. pteronyssinus codon is conservatively replaced with the optimal codon for a mammalian host. Surprisingly such optimised ProDerP1/DerP1 sequences also express very well in yeast despite the different codon usage of yeast.

A still further aspect of the invention provides a process for the preparation of a mutated DerP1/ProDerP1 protein which process comprises expressing DNA, either codon optimised or not, encoding the said protein in a recombinant host cell and recovering the product.

Although DerP1 is well characterized in terms of its enzymatic activity, allergenicity and gene cloning, heterologous expression of DerP1 has been reported to be problematic (Chapman and Platts-Mills, J Immunol 1980; 125:587-592), probably because this cysteine proteinase is synthesized as a PreProDerP1 precursor. Even more problematic is the expression of DerP1/ProDerP1 sequences wherein cysteine residues involved in the protein conformation have been mutated. Accordingly the present invention further provides a process overcoming all these drawbacks therefore allowing the production of the mutated proteins and the industrial development of therapeutic and prophylactic vaccines to mite allergy.

A substantial amelioration of protein expression has been achieved in E. coli when DerP1/ProDerP1 either mutated or not was expressed as a Maltose Binding Protein (MBP) fusion protein. Accordingly there is provided a process for expressing the mutated ProDerP/DerP1 protein as a MBP fusion protein in E. coli. Furthermore, a substantial amelioration of protein expression in yeast has been surprisingly achieved for the mutated protein even though disulphide bonds are said to be essential for secretion in Pichia pastoris (Takai et al. 2001, Int. Arch. Allergy Immunol. 124, 454-460). This was achieved by re-engineering the polynucleotide sequence which encodes the Dermaphagoides mutated ProDerP/DerP1 protein to fit the codon usage found in highly expressed human genes, thereby also allowing the recombinant antigen to have the same conformation and immunological properties as native ProDerP/DerP1 Dermaphagoides allergens. Surprisingly, the cloning and expression of mutated ProDerP1, codon-optimised for mammalian cell expression, could be achieved in Pichia pastoris, with a certain proportion being secreted, although expression in P. pastoris has been formerly reported to be unsuccessful (Takai et al. 2001, Int. Arch. Allergy Immunol. 124, 454-460).

The process of the invention may be performed by conventional recombinant techniques such as described in Maniatis et. al., Molecular Cloning—A Laboratory Manual; Cold Spring Harbor, 1982-1989.

In particular, the process may comprise the steps of:

1. Preparing a replicable or integrating expression vector capable, in a host cell, of expressing a DNA polymer comprising a nucleotide sequence that encodes the said DerP1/ProDerP1 protein;

2. Altering the IgE-binding activity of the resultant protein by replacing the cysteine residues involved in disuphide bonds with another residue, preferably an arginine residue, using site directed mutagenesis;

3. Transforming a host cell with the said vector

4. Culturing the transformed host cell under conditions permitting expression of the DNA polymer to produce the protein; and

5. Recovering the protein.

The term ‘transforming’ is used herein to mean the introduction of foreign DNA into a host cell by transformation, transfection or infection with an appropriate plasmid or viral vector using e.g. conventional techniques as described in Genetic Engineering; Eds. S. M. Kingsman and A. J. Kingsman; Blackwell Scientific Publications; Oxford, England, 1988. The term ‘transformed’ or ‘transformant’ will hereafter apply to the resulting host cell containing and expressing the foreign gene of interest.

The expression vector is novel and also forms part of the invention. One particular aspect of the present invention provides an expression vector which comprises, and is capable of directing the expression of, a polynucleotide sequence encoding a cystein-mutated DerP1/ProDerP1 protein according to the invention. Another particular aspect of the invention provides an expression vector which comprises, and is capable of directing the expression of, a polynucleotide sequence encoding a cysteine-mutated DerP1/ProDerP1 protein wherein the codon usage pattern of the polynucleotide sequence is typical of highly expressed mammalian genes, preferably highly expressed human genes. The vector may be suitable for driving expression of heterologous DNA in bacterial, insect, yeast or mammalian cells, particularly human cells.

The replicable expression vector may be prepared in accordance with the invention, by cleaving a vector compatible with the host cell to provide a linear DNA segment having an intact replicon, and combining said linear segment with one or more DNA molecules which, together with said linear segment encode the desired product, such as the DNA polymer encoding the DerP1/ProDerP1 protein under ligating conditions.

Thus, the DNA polymer may be preformed or formed during the construction of the vector, as desired.

The choice of vector will be determined in part by the host cell, which may be prokaryotic or eukaryotic. Suitable vectors include plasmids, bacteriophages, cosmids and recombinant viruses.

The preparation of the replicable expression vector may be carried out conventionally with appropriate enzymes for restriction, polymerisation and ligation of the DNA, by procedures described in, for example, Maniatis et al cited above. The recombinant host cell is prepared, in accordance with the invention, by transforming a host cell with a replicable expression vector of the invention under transforming conditions. Suitable transforming conditions are conventional and are described in, for example, Maniatis et al cited above, or “DNA Cloning” Vol. II, D. M. Glover ed., IRL Press Ltd, 1985.

The choice of transforming conditions is determined by the host cell. Thus, a bacterial host such as E. coli may be treated with a solution of CaCl₂ (Cohen et al, Proc. Nat. Acad. Sci., 1973, 69, 2110) or with a solution comprising a mixture of RbCl, MnCl₂, potassium acetate and glycerol, and then with 3-[N-morpholino]-propane-sulphonic acid, RbCl and glycerol. Mammalian cells in culture may be transformed by calcium co-precipitation of the vector DNA onto the cells, by lipofection, or by electroporation. Yeast compatible vectors also carry markers that allow the selection of successful transformants by conferring prototrophy to auxotrophic mutants or resistance to heavy metals on wild-type strains. Control sequences for yeast vectors include promoters for glycolytic enzymes (Hess et al., J. Adv. Enzyme Reg. 1968, 7, 149), PHO5 gene encoding acid phosphatase, CUP1 gene, ARG3 gene, GAL genes promoters and synthetic promoter sequences. Other control elements useful in yeast expression are terminators and leader sequences. The leader sequence is particularly useful since it typically encodes a signal peptide comprised of hydrophobic amino acids, which direct the secretion of the protein from the cell. Suitable signal sequences can be encoded by genes for secreted yeast proteins such as the yeast invertase gene and the a-factor gene, acid phosphatase, killer toxin, the a-mating factor gene and recently the heterologous inulinase signal sequence derived from INU1A gene of Kluyveromyces marxianus. Suitable vectors have been developed for expression in Pichia pastoris and Saccharomyces cerevisiae.

A variety of P. pastoris expression vectors are available based on various inducible or constitutive promoters (Cereghino and Cregg, FEMS Microbiol. Rev. 2000,24:45-66). For the production of cytosolic and secreted proteins, the most commonly used P. pastoris vectors contain the very strong and tightly regulated alcohol oxidase (AOX1) promoter. The vectors also contain the P. pastoris histidinol dehydrogenase (HIS4) gene for selection in his4 hosts. Secretion of foreign protein require the presence of a signal sequence and the S. cerevisiae prepro alpha mating factor leader sequence has been widly and successfully used in Pichia expression system. Expression vectors are integrated into the P. pastoris genome to maximize the stability of expression strains. As in S. cerevisiae, cleavage of a P. pastoris expression vector within a sequence shared by the host genome (AOX1 or HIS4) stimulates homologous recombination events that efficiently target integration of the vector to that genomic locus. In general, a recombinant strain that contains multiple integrated copies of an expression cassette can yield more heterologous protein than single-copy strain. The most effective way to obtain high copy number transformants requires the transformation of Pichia recipient strain by the sphaeroplast technique (Cregg et all 1985, Mol.Cell.Biol. 5: 3376-3385).

The invention also extends to a host cell transformed with a replicable expression vector of the invention.

Culturing the transformed host cell under conditions permitting expression of the DNA polymer is carried out conventionally, as described in, for example, Maniatis et al and “DNA Cloning” cited above. Thus, preferably the cell is supplied with nutrient and cultured at a temperature below 45° C.

The product is recovered by conventional methods according to the host cell. Thus, where the host cell is bacterial, such as E. coli it may be lysed physically, chemically or enzymatically and the protein product isolated from the resulting lysate. Where the host cell is mammalian, the product may generally be isolated from the nutrient medium or from cell free extracts. Conventional protein isolation techniques include selective precipitation, absorption chromatography, and affinity chromatography including a monoclonal antibody affinity column.

Alternatively, the expression may be carried out either in insect cells using a suitable vector such as a baculovirus, in transformed drosophila cells, or mammalian CHO cells. The novel protein of the invention may also be expressed in yeast cells as described for the CS protein in EP-A-0 278 941.

Pharmaceutical, immunogenic and vaccine compositions comprising a hypoallergenic DerP1/ProDerP1 derivative according to the invention, or the polynucleotide sequences encoding said proteins, either codon-optimised or not, are also provided. In preferred embodiments the DNA composition comprises a plurality of particles, preferably gold particles, coated with DNA comprising a vector encoding a polynucleotide sequence which encodes a D. pteronyssinus amino acid sequence, wherein the codon usage pattern of the polynucleotide sequence is typical of highly expressed mammalian genes, particularly human genes.

The polynucleotides and encoded polypeptides according to the invention may find use as therapeutic or prophylactic agents. In particlular the polynucleotides of the invention (including a polynucleotide sequence of native ProDerP1—preferably codon optimised) may be used in DNA vaccination (NAVAC), the DNA being administered to the mammal e.g. human to be vaccinated. The nucleic acid, such as RNA or DNA, preferably DNA, is provided in the form of a vector, such as those described above, which may be expressed in the cells of the mammal. The polynucleotides may be administered by any available technique. For example, the nucleic acid may be introduced by needle injection, preferably intradermally, subcutaneously or intramuscularly. Alternatively, the nucleic acid may be delivered directly into the skin using a nucleic acid delivery device such as particle-mediated DNA delivery (PMDD). In this method, inert particles (such as gold beads) are coated with a nucleic acid, and are accelerated at speeds sufficient to enable them to penetrate a surface of a recipient (e.g. skin), for example by means of discharge under high pressure from a projecting device. (Particles coated with a nucleic acid molecule of the present invention are within the scope of the present invention, as are delivery devices loaded with such particles).

Suitable techniques for introducing the naked polynucleotide or vector into a patient include topical application with an appropriate vehicle. The nucleic acid may be administered topically to the skin, or to mucosal surfaces for example by intranasal, oral, intravaginal or intrarectal administration. The naked polynucleotide or vector may be present together with a pharmaceutically acceptable excipient, such as phosphate buffered saline (PBS). DNA uptake may be further facilitated by use of facilitating agents such as bupivacaine, either separately or included in the DNA formulation. Other methods of administering the nucleic acid directly to a recipient include ultrasound, electrical stimulation, electroporation and microseeding which is described in U.S. Pat. No. 5,697,901. Typically the nucleic acid is administered in an amount in the range of 1 pg to 1 mg, preferably 1 pg to 10 μg nucleic acid for particle mediated gene delivery and 10 μg to 1 mg for other routes.

A nucleic acid sequence of the present invention may also be administered by means of specialised delivery vectors useful in gene therapy. Gene therapy approaches are discussed for example by Verme et al, Nature 1997, 389:239-242. Both viral and non-viral vector systems can be used. Viral based systems include retroviral, lentiviral, adenoviral, adeno-associated viral, herpes viral, Canarypox and vaccinia-viral based systems. Non-viral based systems include direct administration of nucleic acids, microsphere encapsulation technology (poly(lactide-co-glycolide) and, liposome-based systems. Viral and non-viral delivery systems may be combined where it is desirable to provide booster injections after an initial vaccination, for example an initial “prime” DNA vaccination using a non-viral vector such as a plasmid followed by one or more “boost” vaccinations using a viral vector or non-viral based system.

In this way, the inventors have found that vaccination with DNA encoding ProDerP1 (preferably codon optimised for mammals) induces a Th1 response in mice models (high titres of specific IgG2a antibodies and low totres of specific IgG1) and, remarkably, the absence of anti-ProDerP1 IgE.

The pharmaceutical compositions of the present invention may include adjuvant compounds, or other substances which may serve to increase the immune response induced by the protein.

The vaccine composition of the invention comprises an immunoprotective amount of the mutated version of the DerP1/ProDerP1 hypoallergenic protein. The term “immunoprotective” refers to the amount necessary to elicit an immune response against a subsequent challenge such that allergic disease is averted or mitigated. In the vaccine of the invention, an aqueous solution of the protein can be used directly. Alternatively, the protein, with or without prior lyophilization, can be mixed, adsorbed, or covalently linked with any of the various known adjuvants.

Suitable adjuvants are commercially available such as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, and chemokines may also be used as adjuvants.

In the formulations of the invention it is preferred that the adjuvant composition induces an immune response predominantly of the TH1 type. High levels of Th1-type cytokines (e.g., IFN-γ, TNFα, IL-2 and IL-12) tend to favour the induction of cell mediated immune responses to an administered antigen. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Accordingly, suitable adjuvants for use in eliciting a predominantly Th1-type response include, for example a combination of monophosphoryl lipid A, preferably 3-de-O-acylated monophosphoryl lipid A (3D-MPL) together with an aluminium salt. Other known adjuvants, which preferentially induce a TH1 type immune response, include CpG containing oligonucleotides. The oligonucleotides are characterised in that the CpG dinucleotide is unmethylated. Such oligonucleotides are well known and are described in, for example WO 96/02555. Immunostimulatory DNA sequences are also described, for example, by Sato et al., Science 273:352, 1996. CpG-containing oligonucleotides may also be used alone or in combination with other adjuvants. For example, an enhanced system involves the combination of a CpG-containing oligonucleotide and a saponin derivative particularly the combination of CpG and QS21 as disclosed in WO 00/09159 and WO 00/62800. Preferably the formulation additionally comprises an oil in water emulsion and/or tocopherol.

Another preferred adjuvant is a saponin, preferably QS21 (Aquila Biopharmaceuticals Inc., Framingham, Mass.), that may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210.

A particularly potent adjuvant formulation involving QS21 3D-MPL & tocopherol in an oil in water emulsion is described in WO 95/17210 and is a preferred formulation.

Other preferred adjuvants include Montanide ISA 720 (Seppic, France), SAF (Chiron, Calif., United States), ISCOMS (CSL), MF-59 (Chiron), Detox (Ribi, Hamilton, Mont.), RC-529 (Corixa, Hamilton, Mont.) and other aminoalkyl glucosaminide 4-phosphates (AGPs).

Accordingly there is provided an immunogenic composition comprising a DerP1/ProDerP1 hypoallergenic derivative as disclosed herein and an adjuvant, wherein the adjuvant comprises one or more of 3D-MPL, QS21, a CpG oligonucleotide, a polyethylene ether or ester or a combination of two or more of these adjuvants. The DerP1/ProDerP1 hypoallergenic derivative within the immunogenic composition is preferably presented in an oil in water or a water in oil emulsion vehicle.

In a further aspect, the present invention provides a method of making a pharmaceutical composition including the step of mutating one or more cysteine residues involved in disulphide bridge formation, such as Cys4, Cys31, Cys65, Cys71, Cys103 or Cys117. The method further comprises the step of altering the codon usage pattern of a wild-type DerP1/ProDerP1 nucleotide sequence, or creating a polynucleotide sequence synthetically, to produce a sequence having a codon usage pattern typical of highly expressed mammalian genes and encoding a codon-optimised cysteine-mutated ProDerP1/DerP1 amino acid sequence according to the invention. Vaccine preparation is generally described in Vaccine Design (“The subunit and adjuvant approach” (eds. Powell M. F. & Newman M. J). (1995) Plenum Press New York). Encapsulation within liposomes is described by Fullerton, U.S. Pat. No. 4,235,877. Conjugation of proteins to macromolecules is disclosed, for example, by Likhite, U.S. Pat. No. 4,372,945 and Armor et al., U.S. Pat. No. 4,474,757.

The amount of the protein of the present invention present in each vaccine dose is selected as an amount which induces an immunoprotective response without significant, adverse side effects in typical vaccines. Such amount will vary depending upon which specific immunogen is employed and whether or not the vaccine is adjuvanted. Generally, it is expected that each dose will comprise 1-1000 μg of protein, preferably 1-200 μg. An optimal amount for a particular vaccine can be ascertained by standard studies involving observation of antibody titres and other responses in subjects. The vaccines of the present invention may be administered to adults or infants, however, it is preferable to vaccinate individuals soon after birth before the establishment of substantial Th2-type memory responses. Following an initial vaccination, subjects will preferably receive a boost in about 4 weeks, followed by repeated boosts every six months for as long as a risk of allergic responses exists.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

The present invention also provides a process for the production of a vaccine, comprising the steps of purifying a DerP1/ProDerP1 derivative according to the invention or a derivative thereof, by the process disclosed herein and admixing the resulting protein with a suitable adjuvant, diluent or other pharmaceutically acceptable excipient.

The present invention also provides a method for producing a vaccine formulation comprising mixing a protein of the present invention together with a pharmaceutically acceptable excipient.

Another aspect of the invention is the use of a protein or polynucleotide as claimed herein for the manufacture of a vaccine for immunotherapeutically treating a patient susceptible to or suffering from allergy. A method of treating patients susceptible to or suffering from allergy comprising administering to said patients a pharmaceutically active amount of the immunogenic composition disclosed herein is also contemplated by the present invention.

A further aspect of the invention provides a method of preventing or mitigating an allergic disease in man (particularly house dust mite allergy), which method comprises administering to a subject in need thereof an immunogenically effective amount of a mutated allergen of the invention, or of a vaccine in accordance with the invention.

### FIGURE LEGENDS

FIG. 1: IgG and IgE-binding reactivity of denatured ProDerP1 expressed in CHO cells. Immunoplates were coated with 500 ng/well of purified native or denatured ProDerP1 and incubated with sera (diluted 1:8) radioallergosorbent positive to D. pteronyssinus. Bound IgE or IgG were quantitated by incubation with mouse anti-human IgE or IgG and alkaline phosphatase-labelled anti-mouse IgG antibodies, followed by an enzymatic assay. Results are expressed as OD₄₁₀ₙₘ values.

FIG. 2: Correlation between the IgE reactivity of MBP-ProDerP1 and natural DerP. Immunoplates were coated with 500 ng/well of purified DerP or MBP-ProDerP1 and inculated with 95 sera (diluted 1:8) radioallergosorbent positive to D. pteronyssinus. Bound IgE was quantitated by incubation with mouse anti-human IgE and alkaline phosphatase-labelled anti-mouse Ig antibodies, followed by an enzymatic assay. Results are expressed as OD₄₁₀ₙₘ values.

FIG. 3: IgE-binding reactivities of MBP-ProDerP1 mutants, carrying the mutations C4R, C31R and C65R. Immunoplates were coated with 500 ng/well of Wild-type or mutant MBP-ProDerP1 and incubated with a pool of 20 sera (diluted 1:8) radioallergosorbent positive to D. pteronyssinus. Bound IgE was quantitated by incubation with mouse anti-human IgE and alkaline phosphatase-labelled anti-mouse IgG antibodies, followed by an enzymatic assay. Results are expressed as OD₄₁₀ₙₘ values.

FIG. 4: Histamine release activity of allergens. Basophils isolated from the peripheral blood of one allergic donor were stimulated with serial dilutions of diiferent allergens. The histamine released from cells was measured by ELISA. The total amount of histamine in basophils was quantified after cell disruption with the detergent IGEPAL CA-630. Results are shown as the ratio of released histamine by allergens to total histamine.

FIG. 5: schematic representation of the animal model of house dust mite allergy.

The examples which follow are illustrative but not limiting of the invention. Restriction enzymes and other reagents were used substantially in accordance with the vendors' instructions.

### EXAMPLE I

General Procedures

1.—SDS PAGE and Western Blot Analysis

Proteins were analyzed by SDS-PAGE on 12.5% polyacrylamide gels. After electrophoresis, proteins were transferred onto nitrocellulose membranes using a semi-dry transblot system (Bio-Rad). Membranes were saturated for 30 min with 0.5% Instagel (PB Gelatins) in TBS-T (50 mM Tris HCl pH 7.5, 150 mM NaCl, 0.1% Tween 80) and incubated with mouse polyclonal serum raised against denatured or native ProDerP1 diluted in blocking solution (1:5000). Immunoreactive materials were detected using alkaline phosphatase-conjugated goat anti-mouse antibodies (Promega, 1:7500) and 5-bromo,4-chloro,3-indolylphosphate (BCIP, Boehringer)/nitroblue tetrazolium (NBT, Sigma) as substrates.

2.—Glycan Analysis

Carbohydrate analysis was carried out with the Glycan Differenciation Kit (Boehringer) using the following lectins: Galanthus nivalis agglutinin (GNA), Sambucus nigra agglutinin (SNA), Maackia amurensis agglutinin (MAA), Peanut agglutinin (PNA) and Datura stramonium agglutinin (DSA). Briefly, purified proteins were transferred from SDS-PAGE onto nitrocellulose membranes. Membranes were incubated with the different lectins conjugated to digoxigenin. Complexes were detected with anti-digoxigenin antibodies conjugated to alkaline phosphatase.

3.—Enzymatic Assays

Enzymatic assays were performed in 50 mM Tris-HCl pH 7, containing 1 mM EDTA and 20 mM L-cysteine at 25° C. in a total volume of 1 ml. Hydrolysis of Cbz-Phe-Arg-7-amino-4-methylcoumarin (Cbz-Phe-Arg-AMC) and Boc-Gln-Ala-Arg-7-amino-4-methylcoumarin (Boc-Gln-Ala-Arg-AMC) (Sigma) (both substrates at a final concentration of 100 μM) was monitored using a SLM 8000 spectrofluorimeter with λₑₓ=380 nm and λₑₘ=460 nm. Assays were started by addition of cysteine activated allergen to a final concentration of 100 nM. Before any assay, purified DerP1 or ProDerP1 was incubated with a mixture of aprotinin- and p-aminobenzamidine-agarose resins (Sigma) to remove any putative trace of serine protease activity.

4.—Protein Determination

Total protein concentration was determined by the bicinchoninic acid procedure (MicroBCA, Pierce) with bovine serum albumin as standard.

5.—DerP1 ELISA

DerP1 or recProDerP1 was detected with an ELISA kit using DerP1 specific monoclonal antibodies 5H8 and 4C1 (Indoor Biotechnologies). The DerP1 standard (UVA 93/03) used in the assay was at a concentration of 2.5 μg/ml.

6.—IgE-Binding Activity

Immunoplates were coated overnight with DerP1 or ProDerP1 (500 ng/well) at 4° C. Plates were then washed 5 times with 100 μl per well of TBS-Tween buffer (50 mM Tris-HCl pH 7.5, 150 mM NaCl, 0.1% Tween 80) and saturated for 1 hr at 37° C. with 150 μl of the same buffer supplemented with 1% BSA. Sera from allergic patients to D. pteronyssinus and diluted at 1/8 were then incubated for 1 hr at 37° C. Out of the 95 sera used in the experiments, 16 sera ranged in their specific anti-D. pteronyssinus IgE values (RAST assays) from 58.1 kU/L to 99 kU/L and 79 above the upper cut-off value of 100 kU/L. Plates were washed 5 times with TBS-Tween buffer and the allergen-IgE complexes were detected after incubation with a mouse anti-human IgE antibody (Southern Biotechnology Associates) and a goat anti-mouse IgG antibody coupled to alkaline phosphatase (dilution 1/7500 in TBS-Tween buffer, Promega). The enzymatic activity was measured using the p-nitrophenylphosphate substrate (Sigma) dissolved in diethanolamine buffer (pH 9.8). OD₄₁₀ₙₘ was measured in a Biorad Novapath ELISA reader.

For IgE inhibition assays, plates were coated with DerP1 or ProDerP1 at the same concentration (0.12 μM). A pool of 20 human sera from allergic patients (RAST value >100 kU/L) was preincubated overnight at 4° C. with various concentrations (3.6-0.002 μM) of DerP1 or recProDerP1 as inhibitors and added on ELISA plates. IgE-binding was detected as described above.

7.—Histamine Release

The histamine release was assayed using leukocytes from the peripheral heparinized blood of an allergic donor and by the Histamine-ELISA kit (Immunotech). Basophils were incubated with serial dilutions of recProDerP1 or DerP1 for 30 min at 37° C. The total amount of histamine in basophils was quantified after cell disruption with the detergent IGEPAL CA-630 (Sigma).

8.—ProDerP1 Denaturation

Recombinant ProDerP1 was heat-denatured for 5 min at 100° C. in presence of 50 mM β-mercaptoethanol.

9.—Immunisations

Groups of ten CBA/J mice (six weeks old) were four weekly immunised with 5 μg of different proteins or 100 μg of different plasmidic DNA. The purified allergens were injected in presence of alum as adjuvant. As controls, groups of mice were immunised with alum or pJW4304 DNA vector. Mice were bled from the retro-orbital venous plexus on days 7, 14, 21, 28 and sera were collected.

10.—Bronchoprovocation

Within 72 h after immunisations, all mice were placed in a Plexiglas chamber (13×19×37.5 cm) and exposed to aerosolised crude D.pteronyssinus extract over a 20-min period for 7 consecutive days. The concentration of crude mite extract was 300 μg/ml. The aerosols were generated by an ultrasonic nebulizer (Syst′ AM). The output of the nebulizer was 0.5 ml/min and the mean particle size of the aerosol was between 1 and 5 μm. As control, mice were nebulized with PBS.

11.—Measurement of DerP1-specific IgG, IgG1 and IgG2a

Sera were assayed for anti-DerP1 IgG, IgG1 and IgG2a antibodies by ELISA. Immunoplates were coated with ProDerP1 (500 ng/well), for 16 hrs at 4° C. Plates were washed 5 times with TBS-Tween (50 mM Tris-HCl pH 7.5, 150 mM NaCl, 0.1% Tween 80) and saturated for 1 hr at 37° C. with 150 μl of the same buffer supplemented with 1% BSA. Serial dilutions of sera in saturation buffer were incubated for 1 hr at 37° C. Plates were washed 5 times with TBS-Tween buffer and antigen-bound antibodies were detected with the second antibody (goat anti-mouse IgG, Promega, USA) coupled to alkaline phosphatase (dilution 1/7500 in TBS-Tween buffer). The enzymatic activity was measured using the p-nitrophenylphosphate substrate (Sigma) dissolved in diethanolamine buffer (pH 9.8). OD₄₁₅ₙₘ was measured in a Biorad Novapath ELISA reader.

Mouse antibody subclass was determined using immunoplates coated as described above and IgG1- or IgG2a-specific biotin-labelled monoclonal antibodies (rat anti-mouse, dilution 1/7000 in TBS-Tween buffer and 1% BSA, Biosource) as second antibodies. Phosphatase alkaline-conjugated streptavidin (1/1000 dilution, Amersham) was added to each well. Assay of the enzymatic activity proceeded as described above.

In all cases, ELISA titers were identified as the reciprocal of the dilution giving a signal corresponding to 50% of the maximal O.D.₄₁₅ value.

12.—Measurement of DerP1-specific IgE

Immunoplates were coated with rat anti-mouse IgE (10 ng/well), for 16 hrs at 4° C. Plates were washed 5 times with TBS-Tween (50 mM Tris-HCl pH 7.5, 150 mM NaCl, 0.1% Tween 80) and saturated for 1 hr at 37° C. with 150 μl of the same buffer supplemented with 1% BSA. Serial dilutions of sera in saturation buffer were incubated for 1 hr at 37° C. ProDerP1 was then added at 500 ng/ml in saturation buffer. Bound ProDerP1 was detected by addition of biotinylated anti-DerP1 monoclonal antibody 4C1 (Indoor Biotechnologies) Plates were washed 5 times with TBS-Tween buffer and antibodies-bound antigen were detected with addition of streptavidin coupled to alkaline phosphatase (dilution 1/7500 in TBS-Tween buffer). The enzymatic activity was measured using the p-nitrophenylphosphate substrate (Sigma) dissolved in diethanolamine buffer (pH 9.8). OD₄₁₅ₙₘ was measured in a Biorad Novapath ELISA reader.

13.—Proliferation Assays

To measure DerP 1-specific T-cell proliferative response, immunised mice were sacrificed before and after bronchoprovocations. Lymphocytes were isolated from spleens. Cells (4×10⁵/well in triplicate), cultured in RPMI 1640 with 10% FCS containing 15 mM HEPES and 30 μM β-mercaptoethanol, were stimulated with serial dilutions of crude mite extract or ProDerP1 in 96-well plates (10 base 2 dilutions of the antigen were tested, starting from a concentration of 25 μg/ml). As control, cells were incubated with only RPMI medium. After 4 days, cells were pulsed with 1 μCi/well [³H] thymidine (Amersham) for 16 hours. Cells were harvested and ³H-thymidine uptake was measured by scintillation counting. Proliferative responses were calculated as the means of quadruplicate wells and were expressed as stimulation index (SI). A stimulation index of >2 was considered positive.

14.—Cytokines Assay

The level of IFNγ and IL-5 in the lymphocyte culture supernatants were measured in ELISA assays. Plates were coated with 1 μg/ml of anti-mouse IL-5 monoclonal (PharMingen) or anti-mouse IFNγ (Biosource) polyclonal antibodies. Plates were washed 5 times with TBS-Tween and saturated for 1 hr at 37° C. with 150 μl of TBS-Tween-BSA. Serial dilutions of splenocyte culture supernatants were added and incubated for 90 min at 37° C. Biotinylated anti-mouse IL-5 (PharMingen, 1 μg/ml) or anti-mouse IFNγ (Biosource, 0.2 μg/ml) antibodies were applied to the plates for 1 h at 37° C. The antigen-antibody complexes were detected by incubation with streptavidin coupled to horseradish peroxydase (dilution 1/10000, Amersham). The enzymatic activity was measured using tetramethylbenzidine (TMB) as substrate (Sigma). The absorbance at 460 nm was measured in a Biorad Novapath ELISA reader. Cytoline concentrations were determined by interpolation from a standard curve performed with purified mouse IL-5 or IFNγ.

15.—Bronchoalveolar Lavage

Three days after the final aerosol exposure, mice were bled and sacrificed. The lungs were immediately washed via the trachea cannula with 1 ml Hank's balanced salt solution (HBSS) which was instilled and gently recovered by aspiration three times. The lavage fluid was centrifuged at 400 g for 10 min at 4° C. The cell pellet was resuspended in 300 μl Hank's balanced salt solution (HBSS) and cells were counted in a Thoma hemocytometer. Cytospin preparations from 501 μl-aliquots were stained with May-Grünwald Giemsa's stain for differential cell counts.

### EXAMPLE II

Expression of MBP-ProDerP1 in E. coli

1.—Construction of MBP-ProDerP1 Expression Vector

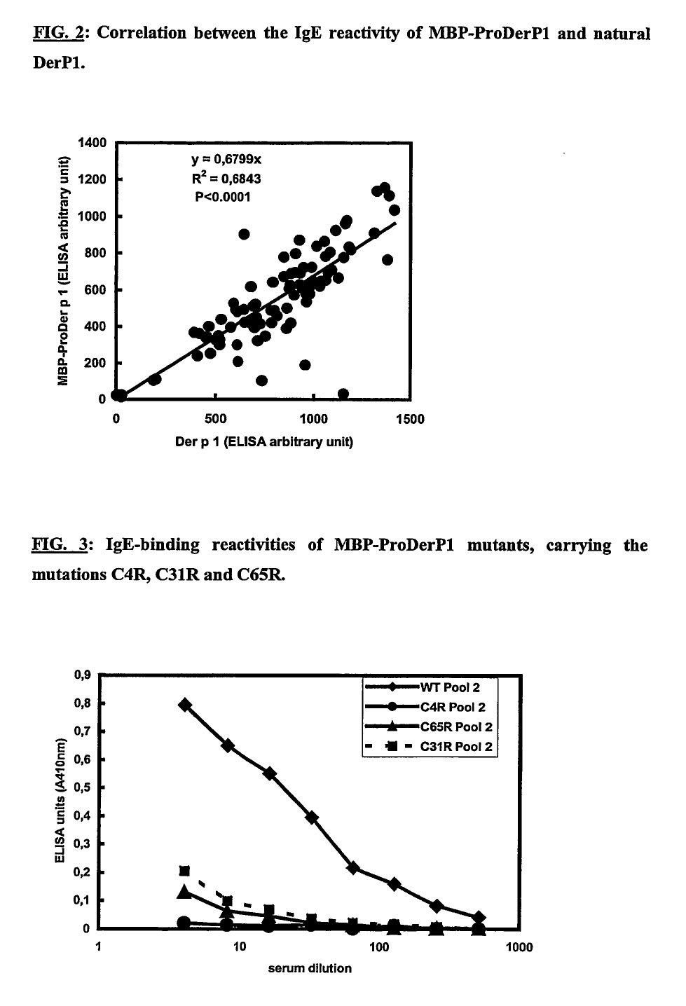

The complete synthetic cDNA encoding ProDerP1 (1-302 aa) (SEQ ID NO:1) was isolated from the eukaryotic expression plasmid pNIV 4846 (a pEE 14-derived expression plasmid carrying humanized ProDerP1 coding cassette, (M. Massaer et al., International Archives of Allergy and Immunology, 2001, 125:32-43) after digestions with Eag I and Xba I. DNA was blunted using large fragment DNA polymerase (Klenow) before Xba I restriction. The 921 bp fragment was inserted at the Asp 718 (blunted end)-Xba I site of pMAL-c2E (New England Biolabs) to give pNIV4854, downstream of the MBP gene. The amino acid sequence of ProDerP1, encoded by the cDNA of SEQ ID NO:1, is represented in FIG. 2 (SEQ ID NO:2).

2.—Site-Directed Mutagenesis

Mutagenesis of DerP1 cysteine residues at position 4, 31 or 65 (mature ProDerP1 numbering, corresponds to positions 84, 111 or 145 in ProDerP1) was performed in the plasmid pNIV4854, after the substitution of DNA fragments carrying one of the three cysteine codons by synthetic oligonucleotides containing the mutations. The following oligonucleotides were used:

5′TTAAGACCCAGTTTGATCTCAACGCGGAGACCAACGCCCGTATCAACGGCA ATGCCCCCGCTGAGATTGATCTGCGCCAGATGAGGACCGTGACTCCCATCCG CATGC3′ (forward) and 5′CGGATGGGAGTCACGGTCCTCATCTG GCGCAGATCAATCTCAGCGGGGGCATTGCCGTTGATACTACGGGCGTTGGTC TCCGCGTTGAGATCGAAACTGGGTC3′ (reverse) to generate a 110 bp Afl II-Sph I fragment for the mutation of cysteine residue 4 to arginine (C4R),

5′CAAGGCGGCCGTGGGTCTTGTTGGGCCTTTCAGGCGTGGCCGCGACAG AGTCGGCATACCTCGCGTATCGGAATCAGAGCCTGGACCTCGC3′ (forward) and

5′TCAGCGAGGTCCAGG CTCTGATTCCGATACGCGAGGTATGCCGACT CTGTCGCGGCCACGCCTGAAAAGGCCCAACAAGACCCACGGCCGCCTTGCAT G3′ (reverse) to generate a 98 bp Sph I-Blp I fragment for the mutation of cysteine residue 31 to arginine (C31R), 5′TGAGCAGGAGCTCGTTGACCGTGCCTCC CAACACGGATGTCATGGGGATACGATTCCCAGAGGTATCGAATACATCCAGC ATA3′ (forward) and 5′CTGGATGTATTCGATACCTCTGGGAATCGTAT CC CCCATGACATCCGTGTTGGGAGGCACGGTCAACGCGCTCCTGC3′ (reverse) to generate a 82 bp Afl II-Sph I fragment for the mutation of cysteine residue 65 to arginine (C65R).

The resulting plasmids containing the ProDerP1 cassette downstream to the MBP gene and carrying respectively the mutations C4R, C31R and C65R were called pNIV4870, pNIV4871 and pNIV4872. All the three mutations were verified by DNA sequencing. Mutated ProDerP1 amino acid sequences respectively carrying C4R, C31R and C65R mutation are illustrated in SEQ ID NO:3, SEQ ID NO:5 and SEQ ID NO:7 respectively. The corresponding encoding nucleic acid sequences are shown in SEQ ID NO:4 (C4R mutation), SEQ ID NO:6 (C31R mutation) and SEQ ID NO:8 (C65R mutation).

3.—Expression and Purification of Wild-Type and Mutant MBP-ProDerP1

E. coli containing the different recombinant expression vectors were grown overnight at 37° C. in 869 medium (A. Jacquet et al., Prot. Exp. Purif. 1999, 17, 392-400) with 100 μg/ml ampicillin. Cells were then diluted 1:100 and allowed to grow at 37° C. to an optical density between 0.4 and 0.6 at 600 nm. Isopropyl β-D-thiogalactoside (IPTG) was added to a final concentration of 0.3 mM. After a 2 h period of induction, cells were harvested by centrifugation at 10000 rpm for 15 min.

Bacterial cell pellets from 1 liter cultures were resuspended in 20 mM Tris-HCl pH 7.5, containing 1 mM aprotinin and AEBSF, and broken under a pressure of 1800 bars using a Cell disrupter (Constant Systems Ltd, Warwick, UK). The lysate was ultracentrifugated at 150,000 g for 60 min. The pellet resulting from the ultracentrifugation was washed with 20 mM Tris-HCl pH 7.5. Insoluble proteins were extracted overnight at 4° C. with 20 mM Tris-HCl pH 7.5 containing 6M urea. The suspension was ultracentrifugated at 150,000 g for 60 min. The supernatant was directly dialysed overnight against 20 mM Tris-HCl pH 7.5, 200 mM NaCl, 1 mM EDTA. The solution was centrifugated to remove any precipitated protein and directly applied onto an amylose resin (1×15 cm) equilibrated in the same buffer. The column was washed with the starting buffer until the A₂₈₀ₙₘ reached the baseline. Proteins were eluted by the addition of 10 mM maltose in the column buffer. Fractions containing the fusion proteins were pooled and concentrated. Purified proteins were stored at −20° C.

### EXAMPLE III

Expression of Three Different ProDerP1 Mutants in CHO Cells

1.—Site-Directed Mutagenesis

Mutations of DerP1 cysteine residues at position 4, 31 or 65 (mature DerP1 numbering, corresponds to positions 84, 111 or 145 in ProDerP1) were introduced into the plasmid pNIV4846. Plasmids pNIV4870, pNIV4871 and pNIV4872, containing the DerP1 cassette downstream to the MBP (see Example II) gene and carrying respectively the mutations C4R, C31R and C65R were each restricted with SfuI-XhoI to isolate a 714 bp fragment. The purified DNA fragments were inserted into plasmid p4846 previously cleaved with the same restriction enzymes. The resulting plasmids containing the DerP1 variants C4R, C31R and C65R were called pNIV4873, pNIV4875 and pNIV4874.

2.—Transient Transfections and Selection of ProDerP1-Producing Stable CHO-K1 Lines.

To determine the production of DerP1 by plasmids pNIV4873, pNIV4875 and pNIV4874, COS cells were transiently transfected by lipofection. For stable DerP1 expression, CH0-K1 cells were transfected with the different plasmids by lipofection. After a 3-weeks 25 μM methionylsulphoximin (MSX) selection, one round of gene amplification was carried out with 100 μM MSX.

### EXAMPLE IV

Denatured ProDerP1 Displays IgG but not IgE-Binding Reactivity Towards Allergic Sera.

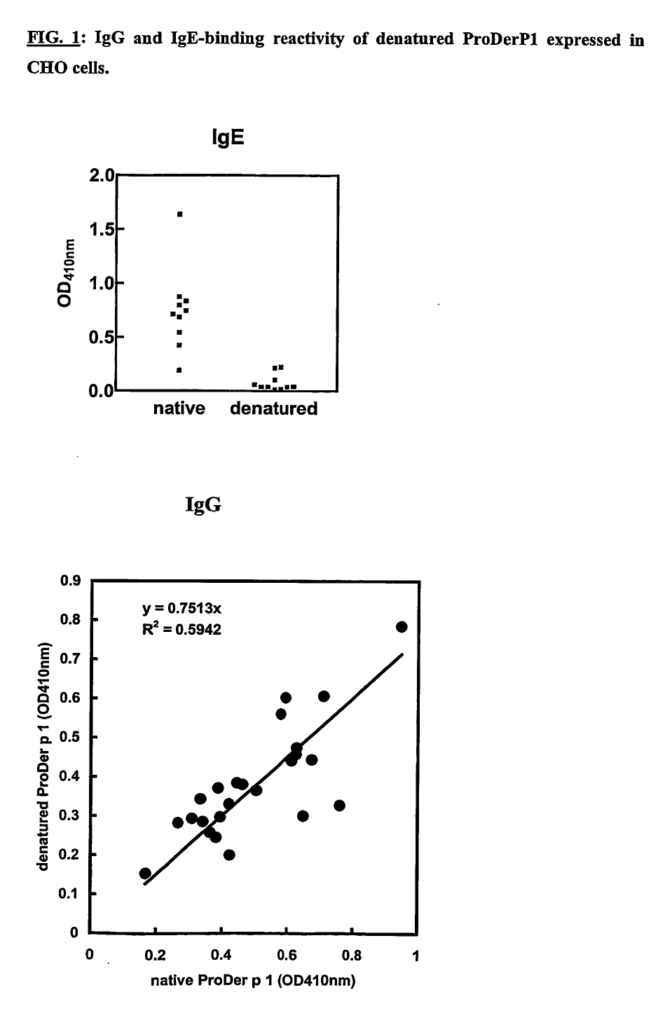

To determine whether a denatured form of ProDerP1 could be used as a hypoallergenic vaccine, IgG- and IgE binding reactivities of denatured (5 min at 100° C. in the presence of 50 mM β-mercaptoethanol) ProDerP1 were assayed in ELISA tests. As shown in FIG. 1, denatured ProDerP1 conserved the main part of the IgG epitopes present on native ProDerP1. On the other hand, the denatured allergen highly lost its IgE-binding reactivity. Our data suggest that denatured ProDerP1 could represent a hypoallergenic variant of ProDerP1.

### EXAMPLE V

IgE Reactivities of MBP-ProDerP1.

The aim of the experiment was to compare the IgE reactivity of MBP-ProDerP1 and of natural DerP1. The reactivity of MBP-ProDerP1 with specific IgE from sera of allergic patients was assessed in a direct ELISA wherein immunoplates were directly coated with DerP1 or MBP-ProDerP1. FIG. 2 shows a strong correlation between the IgE binding to DerP1 and MBP-ProDerP1.

### EXAMPLE VI

IgE-Binding Reactivities of MBP-ProDerP1 Mutants.

The IgE-binding capacity of MBP-ProDerP1 mutants was determined in direct ELISA assays for which immunoplates were directly coated with the different forms of MBP-ProDerP1. A serum pool, made from 20 individual D. pteronyssinus-allergic patient sera with RAST value >100 kU/L, were used in the assays. As shown in FIG. 3, the IgE binding reactivity of the variants C31R and C65R drastically decreased to 5% compared with that of wild-type MBP-ProDerP1. Strikingly, no reactivity (0% left) of IgE to MBP-ProDerP1 was observed when residue cysteine 4 was mutated to arginine. The IgE reactivities were specific of the ProDerP1 moiety as there were no IgE-mediated immune recognitions of MBP or MBP in fusion with an irrelevant protein. Similar results were obtained with another serum pool from 20 others patients.

### EXAMPLE VII

Histamine Release Activity of Various forms of ProDerP1.

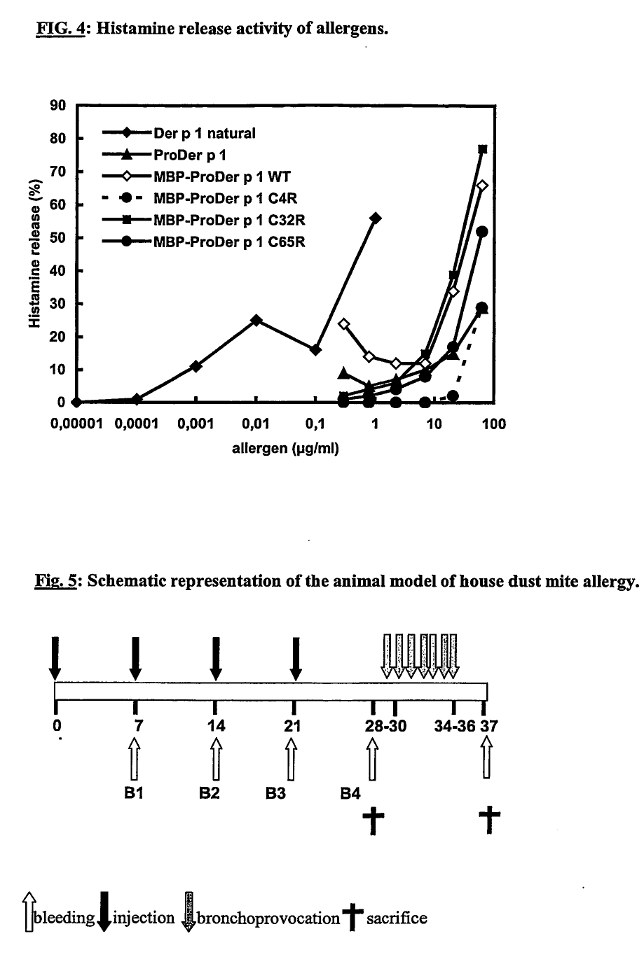

To compare the allergenic activity of natural DerP1 with that of recombinant mutated derivatives of ProDerP1, basophils from one allergic patient were challenged in vitro with various concentrations of allergens and the released histamine was measured. As shown in FIG. 4, natural DerP1 was able to induce histamine release from basophils even at a concentration of 1 ng/ml. By contrast, recombinant mutated forms of ProDerP1 could only release histamine at a 1000-10000-fold higher concentration, These results clearly showed that ProDerP1 mutants display lower IgE binding reactivity than does the natural DerP1.

### EXAMPLE VIII

Immunogenicity Experiments with Various forms of ProDerP1.

1.—Animal Model of House Dust Mite Allergy

An animal model of house dust mite allergy has been developed. CBA/J mice were injected with purified DerP1 adjuvanted with alum. After four injections at one week interval, animals were subjected to a series of bronchoprovocation with D. pteronyssinus extract (FIG. 5). This model was used to test different recombinant forms of DerP1 as well as different DNA as prophylactic vaccines against house dust mite allergy.

2.—Vaccine Formulations

TABLE 1
protein and DNA vaccine formulations tested in the house
dust mite allergy animal model depicted in FIG. 5.

| Protein | DNA | Adjuvant | Way of injection |
|---|---|---|---|
| Natural DerP1 | | Alum | IP |
| ProDerP1 native | | Alum | IP |
| ProDerP1 native | | — | IM |
| ProDerP1 denatured | | Alum | IP |
| MBP-ProDerP1 | | Alum | IP |
| MBP-ProDerP1 C4R | | Alum | IP |
| MBP-ProDerP1 C31R | | Alum | IP |
| MBP-ProDerP1 C65R | | Alum | IP |

IP = intraperitoneal injection
IM = intramuscular injection

3.—Antibody Response—Results

Mice immunized by four injections of natural DerP1 produced high titers of IgG and IgG1, low titers of IgG2a and large amounts of IgE antibodies, indicating that natural DerP1 induces strong Th2 immunes responses (Tables 2 and 4).

The anti-DerP1 IgG and IgG1 antibody responses were also strong in mice injected with native or denatured ProDerP1. After injections with native ProDerP1, the IgG2a titers were slightly higher than those obtained with DerP1, IgE titers being comparable or slightly lower than those obtained with DerP1. In contrast to the native ProDerP1-immunized mice, animals injected with denatured ProDerP1 produced high IgG2a titers and very low IgE antibodies. As expected, immunizations with ProDerP1 in the absence of Alum induced poor immune responses (Table 4).

MBP-ProDerP1 wild type (WT), C4R, C31R and C65R-sensitized mice showed similar productions of specific IgG and IgG1 antibodies (Table 3). Highest IgG2a titers were observed in groups immunized with MBP-ProDerP1 WT and C31R.

Specific IgE titers were low, whatever the MBP-ProDerP1 variants injected.

Similar results were obtained after mice immunizations with plasmid encoding ProDerP1.

Table 2: Titers of specific anti-DerP1 antibodies from mice immunized with different antigens. For IgE titers, results are expressed as OD₄₁₅ₙₘ values for a 1/10 dilution of sera. Titers were also measured after bronchoprovocations with PBS or with D. pteronyssinus (HDM).

| Antigen | Bleeding | Challenge | IgG | IgG1 | IgG2a | IgE |
|---|---|---|---|---|---|---|
| DerP1 | 1 | | <50 | <50 | <50 | 0 |
| | 2 | | 214 | 900 | <50 | 1.1 |
| | 3 | | 700 | 6062 | <50 | 0.2 |
| | 4 | | 2500 | 24390 | 100 | 0.6 |
| | 5 | PBS | 8670 | 16340 | 300 | 0.7 |
| | | HDM | 8230 | 17440 | 300 | 0.6 |
| ProDerP1 | 1 | | <50 | <50 | <50 | 0 |
| native | | | | | | |
| | 2 | | 301 | 1146 | <50 | 1.1 |
| | 3 | | 800 | 6860 | 86 | 0.3 |
| | 4 | | 2500 | 28545 | 203 | 0.5 |
| | 5 | PBS | 8266 | 25500 | 600 | 0.3 |
| | | HDM | 11880 | 38310 | 600 | 0.6 |
| denatured | 1 | | <50 | <50 | <50 | 0 |
| | 2 | | 330 | 861 | 120 | 0.2 |
| | 3 | | 966 | 3402 | 210 | 0.07 |
| | 4 | | 3093 | 14830 | 970 | 0.1 |
| | 5 | PBS | 16380 | 54040 | 2700 | 0.1 |
| | | HDM | 14200 | 32140 | 2700 | 0.05 |

Table 3: Titers of specific anti-DerP1 antibodies from mice immunized with different antigens. For IgE titers, results are expressed as OD₄₁₅ₙₘ values for a 1/10 dilution of sera. Titers were also measured after bronchoprovocations with PBS or with D. pteronyssinus extracts (HDM).

| Antigen | Bleeding | Challenge | IgG | IgG1 | IgG2a | IgE |
|---|---|---|---|---|---|---|
| MBP-ProDerP1 | 2 | | 637 | 3351 | 144 | 0.046 |
| WT | 3 | | 4444 | 24720 | 757 | 0.039 |
| | 4 | | 2500 | 24390 | 100 | 0.6 |
| | 5 | PBS | 6151 | 29500 | 2899 | 0.13 |
| | | HDM | 3437 | 22210 | 1496 | 0.27 |
| MBP-ProDerP1 | 2 | | 583 | 2212 | 95 | 0 |
| C4R | 3 | | 1123 | 6131 | 356 | 0.021 |
| | 4 | | 2500 | 28545 | 203 | 0.5 |
| | 5 | PBS | 2064 | 9077 | 624 | 0.004 |
| | | HDM | 2418 | 14390 | 635 | 0.029 |
| MBP-ProDerP1 | 2 | | 1221 | 4572 | 144 | 0.017 |
| C31R | 3 | | 6472 | 40405 | 1311 | 0.029 |
| | 4 | | 3093 | 14830 | 970 | 0.1 |
| | 5 | PBS | 2897 | 10880 | 857 | 0.063 |
| | | HDM | 5508 | 24300 | 1959 | 0.074 |
| MBP-ProDerP1 | 2 | | 202 | 887 | <50 | 0.022 |
| C65R | 3 | | 1252 | 5718 | 363 | 0.066 |
| | 4 | | 3093 | 14830 | 970 | 0.1 |
| | 5 | PBS | 782 | 3958 | 87 | 0.108 |
| | | HDM | 3109 | 16250 | 430 | 0.117 |

Table 4: Titers of specific anti-DerP1 antibodies from mice immunized with different antigens. For IgE titers, results are expressed as OD₄₁₅ₙₘ values for a 1/10 dilution of sera. Titers were also measured after bronchoprovocations with PBS or with D. pteronyssinus extracts (HDM).

| Antigen | Bleeding | Challenge | IgG | IgG1 | IgG2a | IgE |
|---|---|---|---|---|---|---|
| DerP1 | 2 | | 201 | 1135 | <20 | 0.852 |
| | 3 | | 3264 | 18002 | <50 | 0.34 |
| | 4 | | 8271 | 43306 | <50 | 0.59 |
| | 5 | PBS | 10072 | 57670 | <100 | 0.44 |
| | | HDM | 6058 | 72810 | <100 | 0.68 |
| ProDerP1 | 2 | | 929 | 7422 | 159 | 0.8 |
| Alum | 3 | | 5061 | 27244 | 586 | 0.37 |
| | 4 | | 15110 | 68960 | 1016 | 0.46 |
| | 5 | PBS | 10900 | 57255 | 1190 | 0.421 |
| | | HDM | 16770 | 79460 | 1125 | 0.485 |
| ProDerP1 | 2 | | 136 | 774 | <20 | 0.58 |
| (no adjuvant) | | | | | | |
| | 3 | | 1389 | 8571 | 104 | 0.13 |
| | 4 | | 4704 | 14126 | 120 | 0.17 |
| | 5 | PBS | 3587 | 16930 | 105 | 0.28 |
| | | HDM | 3880 | 20737 | 100 | 0.25 |

4.—T-Cell Proliferative Response—Results

Before (control) and after aerosol challenge, splenocytes isolated from immunized mice were examined for T-cell proliferative response by stimulation with ProDerP1 or D. pteronyssius extract. Results are shown in Table 5 (stimulation index) and in Table 6 (cytokines.).

Allergen-specific T cells responses were detected in immunized mice with the different recombinant ProDerP1 mutants. Strongest responses were observed when splenocytes were restimulated with ProDerP1. T-cell reactivities appeared to be independent from the challenge.

These results in Table 5 indicated that the different forms of ProDerP1 shared common T-cell epitopes with natural DerP1. Moreover, destructuration of ProDerP1 by thermal denaturation or site-directed mutagenesis did not alter ProDerP1 T-cell reactivity, confirming that these forms are hypoallergens with very low IgE-binding reactivity able to stimulated T-cell responses.

TABLE 5
Vaccinated mice were challenged or not with PBS or
D. pteronyssinus extracts. Spleen cells were isolated
and restimulated in vitro with purified ProDerP1 or with
D. pteronyssinus extracts. Stimulation index was measured
by [³H]-thymidine incorporation. —: not available.
These results are obtained from different experiments,
not from only one. Consequently, cytokine assays can
not be compared between all groups.

| | Concen- | | | | | | |
|---|---|---|---|---|---|---|---|
| | tration of | S.I. (stimul. with | | | S.I. (stimul. with | | |
| | stimulating | ProDerP1) | | | HDM ext.) | | |
| | antigen | aerosol | | | aerosol | | |
| Antigen | (μg/ml) | None | PBS | HDM | None | PBS | HDM |
| MBP-ProDerP1 | 50 | 7.3 | 14.97 | 20.8 | — | — | — |
| WT | | | | | | | |
| MBP-ProDerP1 | 50 | 19.1 | 9.7 | 16.3 | — | — | — |
| C4R | | | | | | | |
| MBP-ProDerP1 | 50 | 5.4 | 10.0 | 14.7 | — | — | — |
| C31R | | | | | | | |
| MBP-ProDerP1 | 50 | 6.8 | 8.8 | 13.0 | — | — | — |
| C65R | | | | | | | |
| DerP1 | 40 | — | 1.6 | 17.5 | — | 1.6 | 7.5 |
| ProDerP1 | 40 | — | 30.9 | 11.5 | — | 2.8 | 2.8 |
| ProDerP1 | 40 | — | 24.0 | 15.9 | — | 1.7 | 1.4 |
| denatured | | | | | | | |
| Alum | 40 | — | 4.2 | 4.6 | — | 2.0 | 1.3 |

The presence of cytokines IL-5 and IFNγ in the culture supernatants of restimulated splenocytes was determined in ELISA (Table 6). If we compared the ratio [IFNγ]/[IL-5], we could conclude that vaccinations with natural DerP1 or ProDerP1 adjuvanted with alum induced a better production of IL-5 than IFNγ. The different forms of MBP-ProDerP1 (mutants and wild-type) as well as denatured ProDerP1 induced comparable levels of both cytokines.

Table 6: [IL-5] and [IFNγ] in supernatants from ProDerP1-restimulated splenocytes. These results are obtained from different experiments, not from only one. Consequently, cytokine assays can not be compared between all groups.

| | [IL-5] (pg/ml) | | | [IFNγ] (pg/ml) | | |
|---|---|---|---|---|---|---|
| | Aerosol | | | Aerosol | | |
| Antigen | none | PBS | HDM | None | PBS | HDM |
| MBP-ProDerP1 | 420 | 165 | 929 | 987 | 1076 | 1282 |
| MBP- | 330 | 51 | 308 | 551 | 1366 | 1177 |
| ProDerP1C4R | | | | | | |
| MBP- | 430 | 202 | 1141 | 1348 | 1281 | 3392 |
| ProDerP1C31R | | | | | | |
| MBP- | 0 | 0 | 953 | 0 | 0 | 1161 |
| ProDerP1C65R | | | | | | |
| Alum | 0 | 0 | 0 | 0 | 0 | 0 |
| DerP1 | 75 | 45 | 495 | 0 | 0 | 190 |
| ProDerP1 | 0 | 355 | 400 | 0 | 125 | 210 |
| ProDerP1 | — | 850 | 736 | — | 822 | 1119 |
| denatured | | | | | | |

5.—Bronchoalveolar Lavage—Results

Sensitisation with natural DerP1 and subsequent exposure to aerosolised house dust mite extracts induced significantly higher bronchoalveolar cell numbers (Table 7). Seven exposures to aerosolised house dust mite extracts were shown to induce airway eosinophilia in only the animals vaccinated with DerP1. In this group, airway eosinophilia was not observed when DerP1-sensitised animals were not nebulized or exposed to aerosolised PBS.

Vaccinations with the different recombinant forms of ProDerP1 prevented airway eosinophilia, even after exposure to aerosolised HDM extracts.

Table 7: Characterization of the bronchoalveolar lavage fluid of different antigen-immunized mice exposed to PBS or house dust mite extracts aerosols

| | | | | | | | Total |
|---|---|---|---|---|---|---|---|
| | Aero- | Lympho | Eosino | Neutro | Macro | Mono | cells |
| Antigen | sol | (%) | (%) | (%) | (%) | (%) | (10⁵/ml) |
| DerP1 | none | 86 | 4 | 0 | 6 | 3 | 2.2 |
| | HDM | 13 | 68 | 7 | 6 | 6 | 167 |
| | PBS | 90 | 0 | 2 | 4 | 4 | 4.8 |
| ProDerP1 | none | 90 | 0 | 0 | 7 | 3 | 3.2 |
| | HDM | 69 | 7 | 12 | 3 | 10 | 5.1 |
| | PBS | 76 | 5 | 4 | 7 | 8 | 7.6 |
| ProDerP1 | none | 51 | 5 | 2 | 22 | 20 | 4 |
| denatured | | | | | | | |
| | HDM | 52 | 4 | 26 | 10 | 7 | 6.9 |
| | PBS | 67 | 2 | 2 | 20 | 9 | 5.2 |
| Alum | none | 88 | 1 | 4 | 7 | 0 | 3.6 |
| | HDM | 80 | 0 | 4 | 14 | 1 | 1.5 |
| | PBS | 88 | 1 | 5 | 5 | 1 | 1.2 |
| MBP- | none | 85 | 2 | 4 | 7 | 0 | 1.5 |
| ProDerP1 | | | | | | | |
| | HDM | 70 | 3 | 14 | 8 | 5 | 2.1 |
| | PBS | 88 | 1 | 6 | 5 | 0 | 0.6 |
| MBP- | none | 90 | 2 | 4 | 4 | 1 | 2.2 |
| ProDerP1 | | | | | | | |
| C4R | | | | | | | |
| | HDM | 71 | 2 | 14 | 11 | 1 | 2 |
| | PBS | 80 | 2 | 7 | 10 | 1 | 4.5 |
| MBP- | none | 79 | 1 | 14 | 7 | 0 | 1.3 |
| ProDerP1 | | | | | | | |
| C31R | | | | | | | |
| | HDM | 65 | 4 | 27 | 5 | 1 | 2 |
| | PBS | 87 | 2 | 7 | 5 | 1 | 3 |
| MBP- | none | 85 | 0 | 4 | 10 | 1 | 2.4 |
| ProDerP1 | | | | | | | |
| C65R | | | | | | | |
| | HDM | 84 | 1 | 7 | 7 | 1 | 2.4 |
| | PBS | 84 | 1 | 4 | 12 | 0 | 1.5 |

### EXAMPLE IX

Expression Plasmid for Nucleic Acid Vaccination (NAVAC)

1.—Construction of ProDerP1 Encoding Plasmid for Nucleic Acid Vaccination

The ProDerP1 coding cassette (1-302aa) was excised from plasmid pNIV4846 (see above), restricted with HindIII and BglII, and inserted into plasmid pJW4304 previously cleaved with HindIII and BglII. The resulting plasmid, named pNIV4868, was verified by DNA sequencing.

2.—Site-Directed Mutagenesis

Mutations of ProDerP1 cysteine residues at position 4, 31 or 65 (mature DerP1 numbering, corresponds to positions 84, 111 or 145 in ProDerP1) were introduced into the plasmid pNIV4868. Plasmids pNIV4870, pNIV4871 and pNIV4872, containing the ProDerP1 cassette downstream to the MBP gene and carrying respectively the mutations C4R, C31R and C65R were each restricted with AflII-BamHI to isolate a 699 bp fragment. pNIV 4868 was digested with AflII-HpaI to isolate a 480 bp fragment. The two purified DNA fragments were inserted into plasmid pJW4304 previously cleaved with HpaI-BamHI. The resulting plasmids containing the ProDerP1 variants C4R, C31R and C65R were called pNIV4879, pNIV4880 and pNIV4881.

### EXAMPLE X

Expression of ProDerP1 in Pichia pastoris

1.—Construction of ProDerP1 Expression Vector

The ProDerP1 coding cassette from pNIV4846 (full-length 1-302aa ProDerP1 cDNA with optimised mammalian codon usage) was amplified by PCR using the following primers: 5′ACTGACAGGCCTCGGCCGAGCTCCATTAA3′ (StuI restriction site in bold, forward) and 5′CAGTCACCTAGGTCTAGACTC GAGGGGAT3′ (AvrII restriction site in bold, reverse). The amplified fragment was cloned into the pCR2.1 TOPO cloning vector. The correct ProDerP1 cassette was verified by DNA sequencing. Recombinant TOPO vector was digested with StuI-AvrII to generate a 918 bp fragment which was introduced into the pPIC9K expression vector restricted with SnaBI-AvrII. The resulting plasmid, pNIV4878, contains the ProDerP1 cassette downstream to the S.cerevisae α factor

2.—Site-Directed Mutagenesis

Expression plasmid for the production of unglycosylated ProDerP1 (N52Q, mature DerP1 numbering) was derived from pNIV4878 by overlap extension PCR using a set of four primers. The following primers:

5′GGCTTTCGAACACCTTAAGACCCAG3′ primer 1, AflII restriction site in bold, forward) and 5′GCTCCCTAGCTACGTA TCGGTAATAGC3′ (primer 2, SnaBI restriction site in bold, reverse) were used to amplify a 317 bp fragment encoding the ProDerP1 amino acid sequence 71-176.

The following primers 5′CCTCGCGTATCGGCAACAGAGCCTGGACC3′ (primer 3, mutation N52Q in bold, forward) and 5′GGTCCAGGCTCTGTTGCC GATACGCGAGG3′ (primer 4, mutation N52Q in bold, reverse) were used to introduce mutation N52Q in the ProDerP1 sequence.

The mutated 317 bp AflII-SnaBI fragment was generated by a three-step process. In PCR n°1, primers 1 and 4 were mixed with pNIV4878 to produce a ˜200 bp fragment. In PCR n°2, primers 2 and 3 were mixed with pNIV4878 to produce a ˜140 bp. The two PCR products were purified onto agarose gel and used as templates for a third round of PCR to obtain a ˜340 bp fragment. This purified fragment was cloned into the pCR2.1 TOPO cloning vector (Invitrogen). The mutation was verified by DNA sequencing. Recombinant TOPO vector was digested with AflII-SnaBI to generate a 317 bp fragment which was ligated into the similarly digested pNIV4878. The resulting plasmid, pNIV4883, contains the ProDerP1 N52Q downstream to the S. cerevisae a factor. To obtain unglycosylated variants of ProDerP1 carrying mutations of DerP1 cysteine residues at position 4, 31 or 65 (mature DerP1 numbering), overlap extension PCR using the same set of primers were performed with plasmids pNIV4873, pNIV4875 and pNIV4874. The resulting plasmids pNIV4884, 4885 and 4886 encode respectively ProDerP1 N52Q C4R, N52Q C31R and N52Q C65R.

2.—Transformation of P. pastoris

Plasmid pNIV4878 was introduced into P. pastoris using the spheroplast transformation method. Transformants were selected for histidinol deshydrogenase (His+) prototrophy. The screening of His+transfomants for geneticin (G418) resistance was performed by plating clones on agar containing increasing concentrations of G418.

Transformation with plasmids encoding ProDerP1 N52Q, ProDerP1 N52Q C4R, N52Q C31R and N52Q C65R was performed using the same method.

3.—Production of ProDerP1 by Recombinant Yeast

G418 resistant clones were grown at 30° C. in BMG medium to an OD₆₀₀ₙₘ of 2-6. Cells were collected by centrifugation and resuspended to an OD₆₀ₙₘ of 1 in 100 ml of BMG medium. ProDerP1 expression was induced by daily addition of methanol 0.5% for 6 days. The supernatant was collected by centrifugation and stored at −20° C. until purification.

4.—Purification of ProDerP1 from Yeast Culture Supernatant

Supernatants were diluted 10 times with water and, after pH adjustment to 9, directly loaded onto a Q sepharose column equilibrated in in 20 mM Tris-HCl pH 9. The column was washed with the starting buffer. Protein elutions proceeded by step-wise increasing NaCl concentration in the buffer. The ProDerP1-enriched fractions were pooled and concentrated by ultrafiltration onto a Filtron membrane (Omega serie, cut-off: 10 kD). The ProDerP1 purification was achieved by a gel filtration chromatography onto a superdex-75 column (1×30 cm, Pharmacia) equilibrated in PBS pH 7,3. Purified ProDerP1 was concentrated and stored at −20° C.

1. A recombinant Dermatophagoides pteronyssinus DerP1 or ProDerP1 (DerP1/ProDerP1) protein allergen derivative wherein said allergen derivative has a significantly reduced allergenic activity compared to that the wild-type allergen.

2. A recombinant DerP1/ProDerP1 derivative as claimed in claim 1, wherein said derivative has been thermally treated.

3. A recombinant DerP1/ProDerP1 derivative as claimed in claim 1, wherein said derivative has been genetically mutated.

4. A recombinant DerP1/ProDerP1 mutant as claimed in claim 3, wherein said mutant comprises one or more of the DerP1 following mutation: a mutation of the cysteine 4 residue, a mutation of the cysteine 31 residue, a mutation of the cysteine 65 residue, a mutation of the cysteine 71 residue, a mutation of the cysteine 103 residue and a mutation of the cysteine 117 residue.

5. A recombinant mutant allergen having any of the sequences selected from the group consisting of: SEQ ID NO: 3, SEQ ID NO: 5, SEQ ID NO: 7, SEQ ID NO: 9, SEQ ID NO: 11, SEQ ID NO: 13.

6. An isolated nucleic acid molecule encoding a mutated version of an allergen as claimed in claim 1.

7. A nucleic acid sequence according to claim 6 wherein the codon usage pattern resembles that of highly expressed mammalian genes.

8. An expression vector containing a nucleic acid of claim 6 or 7.

9. A host cell transformed with a nucleic acid sequence of claim 6 or 7.

10. An immunogenic composition comprising a recombinant protein or mutant allergen as claimed in claim 1.

11. An immunogenic composition as claimed in claim 10, wherein the adjuvant is a preferential stimulator of Th1-type immune responses.

12. An immunogenic composition as claimed in claim 10 wherein the adjuvant comprises one or more of 3D-MPL, QS21, a CpG oligonucleotide, a polyethylene ether or ester or a combination of two or more of these adjuvants.

13. An immunogenic composition as claimed in claim 10 wherein the allergen is presented in an oil in water or a water in oil emulsion vehicle.

14. (Cancelled)

15. (Cancelled)

16. A method of treating a patient suffering from or preventing a patient susceptible to allergic responses, comprising administering to said individual an immunogenic composition as claimed in claim 10.

17. An immunogenic composition comprising an encoding polynucleotide as claimed in claim 6, and, optionally, an adjuvant.

18. An immunogenic composition comprising an encoding polynucleotide as claimed in claim 7, and, optionally, an adjuvant.

19. An immunogenic composition comprising an encoding polynucleotide as claimed in claim 8, and, optionally, an adjuvant.

20. An immunogenic composition as claimed in claim 11 wherein the adjuvant comprises one or more of 3D-MPL, QS21, a CpG oligonucleotide, a polyethylene ether or ester or a combination of two or more of these adjuvants.

21. An immunogenic composition as claimed in any of claim 11 wherein the allergen is presented in an oil in water or a water in oil emulsion vehicle.

22. An immunogenic composition as claimed in any of claim 12 wherein the allergen is presented in an oil in water or a water in oil emulsion vehicle.

23. A method of treating a patient suffering from or preventing a patient susceptible to allergic responses, comprising administering to said individual an immunogenic composition as claimed in claim 11.

24. A method of treating a patient suffering from or preventing a patient susceptible to allergic responses, comprising administering to said individual an immunogenic composition as claimed in claim 12.

25. A method of treating a patient suffering from or preventing a patient susceptible to allergic responses, comprising administering to said individual an immunogenic composition as claimed in claim 1.
